(19) United States
(12) Patent Application Publication
(10) Pub. No.: US 2008/0311554 A1
(43) Pub. Date: Dec. 18, 2008

(54) Methods for monitoring patients with severe sepsis and septic shock and for selecting treatments for these patients

(76) Inventor: Gus J. Slotman, Moorestown, NJ (US)

Correspondence Address: Jane Massey Licata;Licata & Tyrrell P.C., 66 E. Main Street, Marlton, NJ, 08053, US

(21) Appl. No.: 12/056,367

(22) Filed: Mar. 27, 2008

Related U.S. Application Data
(63) Continuation-in-part of application No. 11/867,874, filed on Oct. 5, 2007.
Application No. 11/867,874 is a continuation of application No. 10/321,953, filed on Dec. 17, 2002, now Pat. No. 7,297,546.
Application No. 10/321,953 is a continuation-in-part of application No. 09/788,172, filed on Feb. 16, 2001, now abandoned.
Application No. 09/788,172 is a continuation-in-part of application No. 09/139,189, filed on Aug. 25, 1998, now Pat. No. 6,190,872.
Application No. 09/139,189 is a continuation-in-part of application No. 08/612,550, filed on Mar. 8, 1996, now abandoned.
Application No. 08/612,550 is a continuation-in-part of application No. 08/239,328, filed on May 6, 1994, now abandoned.

(51) Int. Cl. G01N 33/50

(52) U.S. Cl. 435/4

(57) ABSTRACT

Methods of identifying, monitoring and matching patients with appropriate treatments who are at risk for developing a systemic inflammatory condition using a systemic mediator-associated physiologic test profile are provided. The methods of the present invention increase the likelihood of demonstrating clinical efficacy in clinical trial datasets.

### INTRODUCTION

The instant patent application is a continuation-in-part of application Ser. No. 11/867,874 filed Oct. 5, 2007, which is a continuation of application Ser. No. 10/321,953 filed Dec. 17, 2002, now issued as U.S. Pat. No. 7,297,546, which is a continuation-in-part of application Ser. No. 09/788,172, filed Feb. 16, 2001, now abandoned, which is a continuation-in-part of application Ser. No. 09/139,189, filed Aug. 25, 1998, now issued as U.S. Pat. No. 6,190,872, which is a continuation-in-part of application Ser. No. 08/612,550, filed Mar. 8, 1996, now abandoned, which is a continuation-in-part of application Ser. No. 08/239,328, filed May 6, 1994, now abandoned, each of which is incorporated herein in its entirety by reference.

### BACKGROUND OF THE INVENTION

Physiologic insults triggering the onset of systemic inflammatory conditions including sepsis, Adult Respiratory Distress Syndrome (ARDS), Systemic Inflammatory Response Syndrome (SIRS) and Multiple Organ Dysfunction Syndrome (MODS) have been identified to include infection and its systemic effects, shock, trauma, inhalation injury, pancreatitis, hypertransfusion, drug overdose, and near-drowning among others. The host response manifested in each of these insults includes increased capillary permeability, organ failure, and death. The mechanism of the response involves diffuse pathologic activation of inflammatory mediators including, but not limited to, endotoxin, leukotrienes B₄, C₄, D₄ and E₄, prostacyclin and thromboxane A₂, activated granulocytes and complement components C3a and C5a, tumor necrosis factor, interleukin-1, interleukin-6, interleukin-8, and other cytokines, neutrophil elastase, platelet activating factor, nitric oxide, and oxide radicals.

Bone, R. C. Annals of Internal Medicine 115:457-469, 1991, reviews the pathogenesis of sepsis and provides a summary of what is known about mediators involved in this pathogenesis along with a hypothesis for understanding how these mediators produce the endothelial dysfunction believed to be one of the key derangements underlying sepsis. Bone (1991) discloses that sepsis and related disorders result in part from endothelial injury caused by repetitive, localized foci of inflammation which, in turn, produce an increase in capillary permeability. Bone suggests that this endothelial dysfunction is the result of the activities of a series of mediators responsible for the pathogenesis. It is proposed that the release of endotoxin or a comparable substance such as enterotoxin, toxic shock syndrome toxin-1, gram-positive or yeast cell-wall products, and viral or fungal antigens, is the initiating event in the sepsis cascade. Once in the circulation, the substance prompts the release of TNF-α, interleukins, and platelet activating factor. Arachidonic acid is then metabolized to produce leukotrienes, thromboxane A₂ and prostaglandins. Almost all of these agents have direct effects on the vascular endothelium. Other suggested agents which may participate in this sepsis cascade include adhesion molecules, kinins, thrombin, myocardial depressant substance, β-endorphin, and heat shock proteins. Bone (1991) presents a pyramid-shaped model of sepsis based upon the theory that the mediators of sepsis can be shown to produce an expanding sequence of events according to the intensity or dose of the original insult. Starting from the top, this pyramid includes (1) infection; (2) release of endotoxin and other bacterial products; (3) release of mediators of inflammation (i.e., cytokines, eicosanoids); (4) sepsis—with or without multi organ failure; (5) sepsis syndrome—with or without multi organ failure; (6) septic shock—with or without multi organ failure; and (7) recovery or death. Bone (1991) suggests that this model may have important implications in the diagnosis and therapy of sepsis.

As a result of identifying causative factors of systemic inflammatory conditions such as sepsis and recent advances in the fields of monoclonal antibodies and recombinant human protein technology, several novel adjuvant treatments have been developed for patients with systemic inflammatory conditions such as sepsis, ARDS, SIRS and MODS. Experimental results and preliminary clinical data suggest that antibodies against gram-negative endotoxin and tumor necrosis factor, human recombinant protein antagonists of interleukin-1 and other cytokines, and inhibitors of platelet activating factor may be beneficial in sepsis, ARDS, MODS and other manifestations of SIRS. Other mediator modifying drugs, such as the cyclo-oxygenase inhibitor ibuprofen, and ketoconazole, a potent antagonist of thromboxane synthetase and 5-lipoxygenase may also be effective in the treatment of ARDS.

Bone, R. C. Clin. Micro. Rev. 6(1):57-68 (1993) provides a review of the epidemiology, diagnosis and current management of gram-negative sepsis and examines the therapeutic potentials of new treatments under development. A variety of physiological changes are disclosed which are associated with the development of sepsis including fever, hypothermia, cardiac manifestations, respiratory signs, renal manifestations and changes in mental status. In addition, important aspects of the effective management of sepsis and a review of current management strategies as well as recent advances including immunotherapy are disclosed.

The promise of these new drugs in the treatment of ARDS, sepsis, MODS and SIRS, however, has not been realized in confirmatory trials following pre-clinical and Phase II testing. One of the primary reasons for these therapeutic failures is the inability of investigators to identify specifically patients most likely to benefit from these treatments at an early stage in the host response, before the pathologic mediator activation that causes the systemic inflammatory response is manifested overtly. Accurate subclinical diagnosis and prediction of organ failure, septic shock and gram-negative infection are even less feasible. Consequently, patients are enrolled in prospective investigations of new treatments for ARDS, sepsis, MODS and SIRS using entry criteria that uniformly reflect late, clinically obvious sequelae of the underlying pathophysiologic processes. Studies of potentially beneficial drugs then fail because patients are enrolled after irreversible tissue damage has occurred, or because so many “at risk” patients must be entered to capture the target population that a drug effect can not be demonstrated, or because the spectra of disease entities and of clinical acuity in the study groups are too variable.

The optimal approach to finding new treatments for ARDS, SIRS, MODS, sepsis and related conditions would be to test new therapeutics in specifically identified patients with high power, accurately predicted risk of developing ARDS, SIRS, MODS, sepsis or a related condition at a time when the acute pathophysiology is still subclinical. Although there are several physiologic scoring systems available which measure the severity of illness, the degree of sepsis, the severity of trauma, or the intensity of organ system dysfunction and are used by physicians to identify certain patient populations, these systems are all based upon obvious, late clinical manifestations of the underlying inflammatory phenomena. The predictive power, accuracy, and specificity of these systems, therefore, are limited.

The Injury Severity Score (ISS) was devised in 1974 as an adaptation of the Abbreviated Injury Scale (AIS). The ISS is a measure of the severity of anatomic injury in victims of blunt trauma and has been found to correlate well with mortality. The score is obtained by summing the squares of the three highest values obtained in five body regions, with 0 points for no injury and 5 points for a critical lesion. The ISS is the most widely used system for grading the severity of an injury; however, it has been criticized as there is a systematic under prediction of death and there is no adjustment for age as a risk factor. The Hospital Trauma Index (HTI) is an adaptation of the ISS which contains both anatomic and physiologic elements in six body regions. A good correlation between ISS, HTI and AIS has been shown.

The Glasgow Coma Scale (GCS) was also introduced in 1974 as a simple, reliable and generally applicable method for assessing and recording altered levels of consciousness. Eye opening, best motor response and best verbal response are monitored and scored independently on a scale ranging from 3 (worst) to 15 (best). The GCS has shown good correlation with functional outcome of survivors and therefore has been incorporated into several other scoring systems.

The Trauma Score (TS) was developed in 1980 for rapid assessment and field triage of injured patients. The TS measures physiologic changes caused by injury. It consists of respiratory and hemodynamic information, combined with the GCS. The TS has been shown to have a high predictability of survival and death.

Physiologic (TS) and anatomic (ISS) characteristics are combined in the TRISS scoring method used to quantify probability of survival following an injury. The method was developed for evaluating trauma care but can be applied to individual patients to estimate the probability of survival.

The Sepsis Severity Score (SSS) was developed in 1983 for grading the severity of surgical sepsis. The system consists of a 6-point scale in seven organ systems including lung, kidney, coagulation, cardiovascular, liver, GI tract and neurologic. The final score is calculated by adding the squares of the highest three values of the three organs with the most severe dysfunction. Studies have shown significantly different scores in survivors versus nonsurvivors and the score correlated well with the length of hospital stay in the survivor group.

The Polytrauma Score (PTS), developed in 1985, is an anatomic injury severity score including an age classification. The score is thought to be more practicable than the ISS while having good correlation with the ISS.

The Multiple Organ Failure Score (MOF score), developed in 1985, grades the function or dysfunction of the seven main organ systems including the pulmonary, cardiovascular, hepatic, renal, central nervous, hematologic, and gastrointestinal systems. This score has been shown to correlate well with mortality outcome.

Also in 1985, APACHE II, a revised version of APACHE (Acute Physiologic And Chronic Health Evaluation) was presented. APACHE II is a disease classification system developed to stratify acutely ill patients admitted to the Intensive Care Unit. Increasing scores have been shown to correlate well with hospital death. The score consists of an acute physiology score (APS), and age score, and a chronic health score. The APS is determined from the most deranged physiologic values during the initial 24 hours after ICU admission. The APACHE system, however, has not consistently predicted mortality risk for trauma patients. APACHE III is the latest revision of APACHE but like its predecessors, the system relies only upon clinically evident data and, therefore, is useful only for predicting mortality risk in selected groups of critically ill patients.

In a study performed by Roumen, R. M. et al., The Journal of Trauma 35(3):349-355, 1993, the relative value of several of these scoring systems in conjunction with measurement of plasma lactate concentration was examined in relation to the development of ARDS, MODS, or both in patients with severe multiple trauma. It was concluded that scoring systems directly grading the severity of groups of trauma patients have predictive value for late and remote complications such as ARDS and MODS, where as scoring systems that grade the physiologic response to trauma, while related to mortality, have no predictive value.

The scoring systems such as APACHE, TRISS, the Sepsis Score and the Multiple Organ Failure Score rely upon overt clinical signs of illnesses and laboratory parameters obtained after the appearance of clinical signs and, thus, are only useful in predicting mortality in a patient.

A study performed on trauma patients at Denver General Hospital in Colorado (Sauaia, A. et al., Arch Surg. 129:39-45, 1994) found that early independent predictors of post-injury multiple organ failure include age greater than 55 years, an Injury Severity Score greater than or equal to 25, and receipt of greater than 6 units of red blood cells in a 12 hour period. Subgroup analysis indicated that base deficit and lactate levels could add substantial predictive value.

Clinical application of any of these prior art scoring systems has been limited to an assessment of grouped percentage risk of mortality. None of the systems are applicable to individual patients. Furthermore, being limited only to predicting risks of hospital death, and possibly consumption of health care resources, the currently variable prognosticated systems can only categorize patients with similar physiology into like mortality risk groups; the systems do not predict important pathophysiologic events in individual patients that could facilitate timely therapeutic intervention and improve survival.

In order for pathophysiologic prognostication to become clinically beneficial to individual patients, a system must predict subclinically the physiologic insults and sequelae of systemic inflammation that lead to mortality in advance so that data-based interventions can be administered in a timely fashion and survival can be optimized. A key to achieving this new level of critical care prediction is to recognize temporal pathophysiology links between baseline clinical and subclinical data and subsequent events in the clinical course of individual patients.

It should be recognized that consensus definitions of sepsis syndrome/severe sepsis/septic shock consistently select critically ill patients with mortality around 35% without shock, and over 40% with shock at baseline. However, these criteria have not identified research subjects whose individual host inflammatory response matched them biologically to the specific drug under study. As a result, Phase III investigations of novel therapies for sepsis have failed to achieve statistically significant treatment effects in improving survival that also were of clinically useful significance. The number of research subjects enrolled in sepsis clinical trials has been increased progressively, powering studies to achieve statistically significant results at the same 3 to 6 percent absolute active drug survival benefits that have been reported all along in smaller previous prior studies. Thus, even the large, statistically successful investigation of an anti-TNF antibody (Pulmonary Reviews.com; Trends in Pulmonary and Critical Care Medicine: Monoclonal antibody improves sepsis. August 2000), and the clinical trial of recombinant activated Protein C (Bernard, et al. 2001. NEJM 344:699-709), while statistically significant, have not achieved survival benefits in sepsis at the clinically valuable levels that are used as standard of care at the bedside.

U.S. Patent Application No. 2003/0211518 describes methods for predicting subclinically, meaning prior to development of signs and symptoms which are diagnostic, a patient's risk for developing a systemic inflammatory condition such as ARDS, SIRS, sepsis and MODS, and predicting their response to a selected therapeutic agent. The methods are based upon predictive models or profiles, referred to as the Systemic Mediator Associated Response Test (SMART), which are generated for a patient and then compared to established baseline values or to a patient's normal values to predict a patient's risk of developing a systemic inflammatory condition and to match the patient with an appropriate treatment for the condition. It has now been found that the SMART methodology can be used to identify patients who will respond with reduced mortality to treatment for severe sepsis and septic shock, specifically treatment with interleukin-1 receptor antagonist. This application of SMART is an extension of the original methodology which was focused on subclinical identification of patients at risk for developing conditions such as sepsis. It must also be appreciated that the use of SMART methodology for enhancement of the design and analysis of clinical trial data is an important development because it increases the likelihood of identifying effective treatments and establishing drug efficacy. Since it is not uncommon for drugs under development to fail in the final stages of clinical efficacy testing, sponsors seek ways to better predict which drugs will be successful. SMART methodology provides sponsors with a way to focus clinical efficacy testing on patients populations most likely to be responsive to a drug's pharmacological effects and thus maximize the likelihood that efficacy will be proven in a clinical trial.

### SUMMARY OF THE INVENTION

An object of the present invention is to provide a method of for analyzing clinical trial results for a new therapeutic agent which comprises measuring baseline parameters of a patient in a clinical trial receiving a new therapeutic agent; measuring baseline parameters of a patient in the clinical trial receiving a placebo; measuring outcome of the clinical trial for the patients of the first two steps; generating, with one or more of the measured parameters, a systemic mediator-associated response test profile for the patients of the first two steps; and predicting, based upon the patient profiles, what the response of each patient to the new therapeutic agent would have been prior to commencing the clinical trial thereby analyzing the clinical trial results of the new therapeutic agent. In a preferred embodiment, the method of the present invention is directed to analyzing clinical trial data for recombinant interleukin-1 receptor antagonist.

### DETAILED DESCRIPTION OF THE INVENTION

The development of systemic inflammatory conditions represents a significant portion of the morbidity and mortality incidence which occur in the intensive care unit (ICU). The term “systemic inflammatory conditions” is used herein to describe conditions which result in a host response manifested by increased capillary permeability, organ failure, and death. Examples of systemic inflammatory conditions include, but are not limited to, ARDS, SIRS, sepsis, MODS, single organ dysfunction, shock, transplant rejection, cancer and trauma. Systemic inflammatory conditions such as ARDS, SIRS and MODS are responsible for more than 70% of the ventilator days spent on the ICU. In addition, ARDS, SIRS, sepsis and MODS are primary causes of death following surgery in surgical ICU patients, thus placing a heavy burden on the health care system.

It is believed that systemic inflammatory conditions, particularly ARDS, SIRS and MODS, are the result of a severe generalized autodestructive inflammation. ARDS is manifested clinically by hypoxemia, hypocapnia, diffuse infiltrates on chest roentgenogram and normal or low left ventricular filling pressures. Circulating prostaglandins, activated complement and abnormal intravascular aggregation of neutrophils have been implicated as possible mediators of ARDS. Slotman et al., Arch Surg. 121:271-274, 1986. Thromboxane B₂ (TxB), prostaglandin 6-keto-F1α (PGI), activated complement components C3a and C5a, and granulocyte aggregation (GA) were found to be significantly elevated in all critically ill patients as compared to normal controls. For patients with ARDS the ratios of TxB/PGI and C3a/C5a also were significantly greater than controls. Differences between patients with and without ARDS in this study, however, were significant only for increased GA and plasma C3a in ARDS.

Circulating prostaglandins, activated complement, and pathologic neutrophil aggregation are also involved in the clinical response to injury and infection and in the hemodynamic dysfunction of septic and hypovolemic shock. PGI, activated complement components C3a and C5a, and GA responses were significantly increased in critically ill patients as compared to normal control values. Slotman, G. J. et al., Surgery 99(6):744-750, 1986. TxB levels were also found to be significantly elevated in patients with severe sepsis and septic shock.

Treatments for systemic inflammatory conditions have failed to reach their full potential as early subclinical identification of appropriate patients to participate in clinical efficacy studies has proven most difficult. Physiologic scoring systems which are used by physicians to predict mortality in a patient have generally proven insufficient in predicting the onset of a systemic inflammatory condition subclinically.

In the present invention a method of identifying patients with severe sepsis and septic shock who will respond with reduced septic mortality to a treatment for sepsis is provided. The method is based on use of treatments that include, but are not limited to, intravenous recombinant interleukin-1 receptor antagonist (IL-1ra). The method comprises generating and comparing a systemic mediator-associated response test (SMART) profile for a patient with an established profile of a clinical population that responded to IL-1ra treatment with reduced mortality to identify whether the patient is likely to respond to treatment with IL-1ra based on the comparison. Accordingly, the present invention meets a long felt need for a method of optimizing treatment and identifying patients most likely to respond to a particular treatment, thereby reducing the likelihood that a patient will fail to respond, and in the case of sepsis this failure to respond is often death. Generally, systemic inflammatory conditions do not develop in healthy individuals but rather in patients with preexisting severe disease or in persons who have suffered catastrophic acute illness or trauma. Patients at greatest risk of dying of a systemic inflammatory condition are the elderly; those receiving immunosuppressive drugs; and those with malignancies, cirrhosis, asplenia, or multiple underlying disorders. Bone, R. C. Annals of Internal Medicine 115:457-469, 1991. Accordingly, optimizing treatment for patients in this high risk group would be especially useful to clinicians. It must also be remembered that many sepsis patients were healthy before undergoing some type of trauma and as such healthy individuals who have undergone severe trauma and are now either at risk of developing sepsis or who have developed sepsis would also benefit from application of the present invention. Once a patient is identified as likely to respond based on comparison of his or her SMART profile to the SMART profile established in the clinical population that responded to treatment, the physician would employ their experience and judgment in determining the appropriate mode and timing of treatment. In the present invention the SMART data shows that IL-1ra is an effective treatment for some patients.

Although the data described are focused on IL-1ra treatment and selection of patients that will respond to this treatment, the method of the present invention can also be used to identify other treatments that could be used successfully to treat patients with severe sepsis or any other similar systemic inflammatory condition. Therefore, methods are also provided for matching patients with other novel treatments based upon comparison of SMART profiles for the patient and established control profiles for effective treatments or new treatments in development. By matching patients with treatments, effective treatments for patients at risk for developing a systemic inflammatory condition can be selected. The optimization of treatment for patient populations with the present method is an improvement on current methods of clinical trial data analysis and increases the likelihood that efficacy will be shown in the clinical trial. In this method, a SMART profile is generated for the patient from selected patient parameters. The patient SMART profile is then compared with established control profiles for effective treatments. Selection of a treatment for the patient is based upon comparing and identifying the established control profiles for effective treatments which exhibit similarities to the patient's profile. In addition, appropriate patient populations for testing of new drugs in development can be selected via matching of patients with treatments based upon SMART profiles. By “appropriate patient population” it is meant subjects who meet the clinical entry criteria of a study for a new drug and who were ready biologically to respond to the new drug if randomized to it.

For purposes of this invention, a “control profile” or “clinical population profile” was generated from a data base containing mean values for selected patient parameters from a population of patients being treated for severe sepsis or septic shock with IL-1ra or placebo, as part of a clinical trial. In other clinical patient populations, a “control profile” can be generated from another data base containing mean values for selected patient parameters from a population of clinical patients with similar conditions and/or injuries or profiles of changing parameters associated with a similar condition and/or injury, or can be generated from the same patient to compare and monitor changes in the patient parameters over time.

By “control profile for effective treatment” it is meant that the control profile, as defined supra, is linked to a treatment identified to be effective in those patients with similar conditions and/or injuries from which the control profile was generated.

SMART profiles of the present invention are generated from one or more patient parameters. Patient parameters, for purposes of this invention, may include selected demographic variables, selected physiologic variables and/or results from selected standard hospital laboratory tests.

Exemplary demographic variables which may be selected for inclusion in a SMART profile include, but are not limited to, age, sex, race, comorbidities such as alcohol abuse, cirrhosis, HIV, dialysis, neutropenia, COPD, solid tumors, hematologic malignancies, chronic renal failure and the admitting service, i.e., surgery or medicine, and trauma.

Examples of physiologic variables which may be selected for inclusion in a SMART profile include, but are not limited to, physical examination, vital signs, hemodynamic measurements and calculations, clinical laboratory tests, concentrations of acute inflammatory response mediators, and endotoxin levels. More specifically, physiologic variables selected may include height, weight, temperature, MAP, heart rate, diastolic blood pressure, systolic blood pressure, mechanical ventilation, respiratory rate, pressure support, PEEP, SVR, cardiac index and/or PCWP of the patient. In addition, complete blood count, platelet count, prothrombin time, partial thromboplastin time, fibrin degradation products and D-dimer, serum creatinine, lactic acid bilirubin, AST, ALT, and/or GGT can be measured. Heart rate, respiratory rate, blood pressure and urine output can also be monitored. A full hemodynamic profile can also recorded in patients with pulmonary artery catheters and arterial blood gases are performed in patients on ventilators. Chest X-rays and bacterial cultures can also performed as clinically indicated. Examples of inflammatory response mediators which can be determined from a biological sample obtained from the patient include, but are not limited to, prostaglandin 6-keto F1α (PGI) (the stable metabolite of prostacyclin), thromboxane B₂ (TxB) (the stable metabolite of thromboxane A₂), leukotrienes B₄, C₄, D₄ and E₄, interleukin-6, interleukin-8, interleukin-1β, tumor necrosis factor, neutrophil elastase, complement components C3 and C5a, platelet activating factor, nitric oxide metabolites and endotoxin levels.

Exemplary hospital laboratory tests considered standard by those skilled in the art which may be selected for inclusion in a SMART profile include, but are not limited to, levels of albumin, alkaline phosphatase, ALT, AST, BUN, calcium, cholesterol, creatinine, GGT, glucose, hematocrit, hemoglobin, MCH, MCV, MCHC, phosphorus, platelet count, potassium, total protein, PT, PTT, RBC, sodium, total bilirubin, triglycerides, uric acid, WBCL, base deficit, pH, PaO₂, SaO₂, FiO₂, chloride, and lactic acid.

Some or all of these patient parameters are preferably determined at baseline (before drug treatment or intervention), and daily thereafter where applicable, and are entered into a database and a SMART profile comprising one or more of the patient parameters is generated from the database. As one of skill in the art will appreciate upon this disclosure, as other additional patient parameters are identified which are predictive of a systemic inflammatory condition, they can also be incorporated into the database and as part of the SMART profile. Similarly, as SMART profiles are generated for more patients and additional data are collected for these parameters, it may be found that some parameters in this list of examples are less predictive than others. Those parameters identified as less predictive in a larger patient population need not be included in all SMART profiles.

Examples of biological samples from which some of these physiologic parameters are determined include, but are not limited to, blood, plasma, serum, urine, bronchioalveolar lavage, sputum, and cerebrospinal fluid.

PGI, TxB, and the leukotrienes B₄, C₄, D₄ and E₄ are derived from polyunsaturated fatty acids via arachidonic acid. These molecules play an important role in smooth muscle contraction, affecting blood pressure, blood flow, the degree of bronchial constriction and uterine contraction. Thromboxane is a potent vasoconstrictor and enhancer of platelet aggregation. Other prostaglandins and the leukotrienes promote the inflammatory response. Leukotrienes act as chemotactic agents, attracting leukocytes to the site of inflammation. Tumor necrosis factor α (TNFα) is a cytokine primarily produced by activated macrophages. TNFα stimulates T-cell and B-cell proliferation and induces expression of adhesion molecules on endothelial cells. This cytokine also plays an important role in host defense to infection. Platelet activating factor mediates platelet homeostasis and interacts with cytokines such as TNFα. Imbalances in PAF can result in uncontrolled bleeding or clot formation and a shock-like hemodynamic and metabolic state. The interleukins 1β, 6, and 8 and complement components C3a and C5a also play a major role in host defense to infection and in the host inflammatory response. Increased cytokine and complement levels in a patient are indicative of an infection and/or inflammation. Neutrophil elastase is an enzyme which hydrolyzes elastin. Elastin is a fibrous mucoprotein that is a major connective tissue protein in tissues with elasticity. Nitric oxide helps to regulate smooth muscle tone possibly through interaction with the prostaglandins and cytokines. The presence of increased nitric oxide metabolites in a biological sample may be indicative of an imbalance in protein degradation or impairment of renal function in a patient. The presence of endotoxin in a biological sample obtained from the patient is indicative of a gram negative bacterial infection. Such infections can lead to the development of shock in a patient. Pathological imbalances of the dynamic equilibrium among these and other biologically active substances cause endothelial damage, increased capillary permeability, and the cascade of subclinical events that leads to systemic inflammatory conditions such as sepsis, ARDS, SIRS, and MODS.

In order to determine levels of these multiple biochemical and cellular inflammatory mediators simultaneously for use in generation of a SMART profile, methods routinely used in automated immunoassay machines are useful. These include, but are not limited to ELISA assays. As will be obvious to those of skill in the art upon this disclosure, however, other means for measuring the selected mediators can also be used.

As will be understood by those of skill in the art upon reading this disclosure, prediction of the patient's risk of developing a systemic inflammatory condition can be based upon a SMART profile which comprises the set of patient parameters discussed supra. Alternatively, these predictions can be based upon a SMART profile comprising only a portion of the patient parameters. Since the patient parameters for each patient's, as well as the control profiles or clinical population profile, are stored in a database, various SMART profiles comprising different patient parameters can be generated for a single patient and compared to an established control profile comprising the same parameters. The ability of these various profiles to be predictive can then be determined via statistical analysis. For example, comparison of a SMART profile comprising only demographics and standard hospital laboratory tests to established control profiles comprising these same parameters has been found to be predictive of risk. See Examples 2 and 3.

Continuous, normally distributed variables are evaluated using analysis of variance. When appropriate, statistical comparisons between subgroups are made using the t-test or the chi-squared equation for categorical variables. Relative risks of developing sepsis or multiple organ failure are computed using a least square regression and logistic regression.

The physician or another individual of skill in the art uses the predictions of the SMART profile as a guide to identifying patients at risk for developing a systemic inflammatory condition prior to development of clinical symptoms of the systemic inflammatory condition. By comparing the SMART profile generated from selected patient parameters, the patient can be categorized by severity of the signs and symptoms, and the presence of systemic inflammatory disease progression or potential development identified.

A first set of experiments was performed where the utility of the SMART methodology was defined based on analysis of the placebo treatment group from a clinical trial for sepsis. This retrospective analysis was performed to determine whether circulating levels of eicosanoid mediators of inflammation, physiologic measurements and standard clinical laboratory results are predictive of inflammation and organ failure in patients with severe sepsis. Seventy-three patients admitted to the Intensive Care Unit and/or Emergency Department who were subsequently diagnosed as septic were studied. Clinical data collected at the time of admission, referred to hereinafter as “baseline” data, included Glasgow Coma Score, systolic, diastolic and mean arterial blood pressures, respiratory rate and urine output. Routine clinical laboratory measurements included serum BUN, creatinine, bilirubin, AST, and arterial blood gas (PaO₂, HCO₃, SaO₂ and PaO₂/FiO₂ ratio). Minimum platelet count and maximum prothrombin time for each 24-hour period was measured also. Vital signs, physical examination and clinical laboratory data were obtained at baseline and daily for days 1-7 and then repeated if the patient was available on days 14, 21 and 28 of observation. In addition, aliquots of blood were collected from each patient at baseline to determine levels of thromboxane B₂ (TXB₂), prostaglandin 6-keto-F1-α (PGI₂), leukotriene B₄ (LTB₄), leukotrienes C₄, D₄, E₄ (LTC₄, LTD₄, LTE₄), interleukin-1β (IL-1), interleukin-6 (IL-6), and tumor necrosis factor (TNF).

For the observed physiologic and clinical laboratory parameters, the most pathologic value noted from baseline through 28 days was determined. Maximum values were collected for Glasgow Coma Score, the Murray Scale for acute respiratory failure, serum creatinine, bilirubin, AST and prothrombin time. The lowest observed measurement was recorded for systolic, diastolic, and mean arterial pressures, platelet count, and arterial blood gas parameters of pO₂, SaO₂, HCO₃ and PaO₂/FiO₂ ratio.

Clinically significant interactions were first determined by pair-wise correlation matrix analysis using, for example, cross correlation analysis, log transformation plus cross correlation and non-parametric cross correlation. The most significant cross correlations were then subjected to linear regression analysis and, finally, multivariate analysis to establish predictive models for survival time, pulmonary dysfunction, renal dysfunction, hepatic dysfunction, cerebral dysfunction and disseminated intravascular coagulation (DIC). Once the predictive model for each indicator was established, its accuracy was tested retrospectively by recalculating a predicted organ function indicator level from its derived equation and plotting these values against the data actually observed. Scores for each parameter in each patient were then ranked and divided into septiles of approximately 10 values each. Then, defining the adult respiratory distress syndrome (ARDS) as a Murray score of 7 or greater, and defining hepatic, renal or cerebral dysfunction and DIC, the percent of the patients in each septile of predicted scores for end-organ failure and for survival time were plotted against the percentage of that septile which developed the target condition at baseline or anytime thereafter, up to 28 days. For survival time, septile of decreasing severity were plotted against survival time in days.

A strong correlation of prothrombin time predicted by the SMART profile versus observed values was evident. A similar strong interaction between predicted creatinine, which includes the log of PGI₂, and observed maximum creatinine levels in septic patients over the creatinine range of 0 to 4 mg/dl was observed. The septiles of increasing creatinine score were also plotted against the percent of patients in each septile who developed acute renal failure from baseline through 28 days and a linear relationship of ascending SMART profile renal failure score and the incidence of subsequent renal failure was observed. Cerebral dysfunction, as defined by a Glasgow Coma Scale of less than 9, was also observed with progressively increasing frequency as the SMART profile score for cerebral dysfunction increased. A linear relationship between predicted and observed SaO₂ was also seen within the physiologically important range of 85 to 100% SaO₂. In addition, a strong relationship between the SMART profile's maximum Murray score and that actually observed in the 73 patients was seen. Over the range of 1 to 12 on the Murray scale, the fit of the predictive equation, which includes PGI₂ and TXB/PGI₂ interactions, among others, has a linear relationship. Over 62% of the variation in the observed Murray values was accounted for by the SMART method. This is most significant, considering the small number of patients involved and the fact that only seven subclinical inflammatory response mediators were measured at baseline.

Further, a direct relationship between decreasing severity of the SMART profile's survival time score with increasing mean survival time was seen thereby demonstrating that, overall, the SMART method indicates not only percentage risk of developing a systemic inflammatory condition but quantitative survival time for the 28 day period after baseline, as well.

Linear regression analysis was also performed for 59 of the 73 patients with sepsis syndrome on whom TXB₂, PGI₂, LTB₄, LTC₄, LTD₄, LTE₄, IL-1, IL-6, and TNF alpha levels, in addition to a completed battery of physiologic indicators of organ failure were measured. Multivariate regression equations were developed using baseline mediator levels and the worst organ system physiology exhibited in each patient. The predicted outcome versus the observed outcome was then plotted for each parameter. The SMART multivariate regression equations account for 17.1% to 90.4% of the variability of each parameter in this study. Statistically, the percent of variation accounted for by SMART equations will increase toward 100% as the total number of patients in each group increases.

The actual values and values predicted by SMART for a number of physiologic parameters in these 59 patients were compared at 24 hours, 48 hours and 72 hours after baseline determination. The predicted values for each patient at 24, 48 and 72 hours were determined by SMART based upon baseline measurements in each patients. Statistically relevant correlations were seen between the predicted values by SMART profiles and the actual values measured.

Accordingly, SMART profiles established a baseline for levels of these parameters in patients having a systemic inflammatory disease such as sepsis and served as a control for comparison in identifying patients at risk for developing the disease. The integration of physiologic variables and subclinical reactants through generation of a SMART profile was also found to be useful in predicting levels of circulating inflammatory mediators in patients. Clinical observations, standard laboratory tests and plasma eicosanoid and cytokine levels recorded prospectively in 24 adults with sepsis syndrome were analyzed retrospectively. Baseline data were used to develop a multivariate regression model that predicted acute inflammatory response mediator blood concentrations up to 72 hours in advance. Predicted plasma levels versus observed measurements for TxB₂, PGI, LTB₄ and LTC₄, LTD₄, LTE₄, IL-1, IL-6 and TNF were compared using linear regression analysis. It was found that predictions made using baseline data correlated well with actual observed levels. Accordingly, the SMART profiles provided a means of prognosticating the course of acute inflammatory mediators in systemic inflammatory conditions. Further validation experiments of the SMART profile system were performed in patients with severe sepsis or septic shock enrolled in a clinical trial. The results of these experiments are presented in Example 2.

SMART profiles were also applied to a database generated from a second phase III clinical trial of the E5 anti-endotoxin antibody with the objective of identifying subjects who met the clinical entry criteria of the study and who were ready biologically to respond to the active E5 if randomized to it. Using multivariate stepwise logistic regression techniques, SMART profiles were developed that predicted which patients were most likely to respond to the active antibody. Baseline data tested included demographics, physiologic observations, hospital laboratory tests, and plasma levels of endotoxin and cytokines. In these experiments, SMART profiles were first developed separately from the placebo and from active E5 baseline databases. Logistic regressions were also developed to determine which independent variables contributed to the dichotomous dependent variables death and organ failure and/or death. The patients were separated by treatment group and one logistic regression model was developed using patients receiving the E5 treatment and a second logistic regression model was developed for the placebo patients. Independent variables for both models were selected by stepwise selection with all ways elimination. Both of the logistic regression models created two possible probabilities for each patient; the probability of survival for the patient receiving E5 and the probability of survival for the patient receiving placebo. Possible cut-offs for the probabilities were examined to determine which patients would have the best survival if they received E5, and which would recover independent of treatment if they received placebo. Examining different cohorts of patients with Kaplan-Meier survival models determined the cut-off. Exploration into the relationships between SMART predictive models and outcomes of E5 and placebo study arm patients resulted in a model that predicted an 80% probability of treatment success for subjects who received E5. In addition, research subjects who had been entered into the E5 study, and who were predicted by the final SMART models to be E5 responders were randomized to placebo and active drug. Kaplan-Meier survival analyses then were performed comparing the results of E5 versus placebo. Treatment effects of E5 on organ/failure death were also analyzed on these same groups.

The first signs of an E5 treatment effect were evidenced by differences in the weighted independent variables for SMART models developed from the E5 versus the placebo databases. In survival modeling, for example, weighted independent variables for the placebo cohort included APACHE II score, urinary tract infection, respiratory tract infection, diastolic blood pressure, and the presence/absence of DIC. The models for the active E5 cohort were quite different, and included APACHE II score, age, neurologic conditions, acute central nervous system dysfunction, ARDS, DIC, and hepatobiliary failure as weighted independent variables. The ROC AUC for the E5 survival model was 0.810, indicating very good prognostic discrimination between outcomes.

Exploration into the relationship between the placebo and active E5 models and their interactions with the treatment effects observed in the two study arms revealed a SMART profile predictive of an 80% probability of treatment success if the patient received E5 and capable of identifying at pre-randomization baseline those subjects who are suited biologically to respond to E5. Baseline data from 759 evaluable patients enrolled in a parent study were then entered into this SMART profile, resulting in a study population of 388 patients who were predicted to respond to E5 if they received active drug. These subjects were then analyzed as placebo or active E5 according to their actual randomization into the parent study. In the parent study (n=759), placebo 30-day mortality was 27.4% and E5 was 26.2%. This was a 1.2% absolute and a 4.4% relative reduction in mortality by E5 (p=0.747). Among the 388 subjects who fit the SMART profile for E5, mortality in the placebo cohort was 17.1%. For the E5 group, mortality was 8.0%. This absolute 9.1% reduction in mortality by E5 translated into a 53.2% relative reduction, statistically significant at the p=0.006 level. In the parent study, 35% had documented gram negative infections. Among the 388 patients in the SMART cohort, 41$ had gram negative infections.

SMART identification of subjects appropriate for E5 beneficially influenced the active drug's effect on organ failure as well. As shown in table 1 below, E5 versus placebo p values for ameliorating organ failure/death were reduced dramatically in the SMART population.

TABLE 1
Analysis of E5 Data Versus Placebo

| P Values | All (n = 759) | SMART (n = 388) |
|---|---|---|
| ARDS | 0.43 | 0.01 |
| Hepatobiliary | 0.65 | 0.03 |
| failure | | |
| Acute renal | 0.81 | 0.22 |
| failure | | |
| Cerebral | 0.20 | 0.02 |
| dysfunction | | |
| DIC | 0.54 | 0.002 |
| Shock | 0.97 | 0.04 |

SMART was also applied to selected patient parameters collected at pre-randomization baseline in two sequential clinical trials, NORASEPT I and NORASEPT II, that tested the efficacy of an antibody against tumor necrosis factor (TNFMab) in patients with severe sepsis and septic shock. SMART profiles were generated from the NORASEPT I database using the following selected patient parameters: APACHE Score, demographics information, vitals at infusion, laboratory results from blood and urine analysis, hematology laboratory results, pulmonary assessment, sepsis, shock episodes, and organ failure. Logistical regressions of the SMART profiles for predicting mortality in patients receiving placebo and patients receiving treatment in the NORASEPT I study are shown below:

TABLE 2
Patients Receiving Placebo/Outcome is Death
on or before 30 days

| | | | Standard | Chi- | |
|---|---|---|---|---|---|
| Parameter | DF | Estimate | Error | Square | Pr > ChiSq |
| Intercept | 1 | −1.8724 | 0.8723 | 4.6077 | 0.0318 |
| APACHE | 1 | 0.0796 | 0.0213 | 13.9415 | 0.0002 |
| B1_Urinary_Tract | 1 | −0.9126 | 0.3031 | 9.0637 | 0.0026 |
| Respiratory | 1 | 0.7521 | 0.4260 | 3.1169 | 0.0775 |
| RESP | 1 | 0.0383 | 0.0154 | 6.1520 | 0.0131 |
| DIAST | 1 | −0.0315 | 0.00939 | 11.2441 | 0.0008 |
| DIC | 1 | 2.0274 | 0.4054 | 25.0156 | <.0001 |

TABLE 3
Odds Ratio Estimate

| | Point | 95% Wald | |
|---|---|---|---|
| Effect | Estimate | Confidence Limits | |
| APACHE | 1.083 | 1.039 | 1.129 |
| B1_Urinary_Tract | 0.0401 | 0.222 | 0.727 |
| Respiratory | 2.121 | 0.920 | 4.889 |
| RESP | 1.039 | 1.008 | 1.071 |
| DIAST | 0.969 | 0.951 | 0.987 |
| DIC | 7.594 | 3.431 | 16.808 |

TABLE 4
Patients Receiving Treatment/Outcome is
Death on or before 30 days

| | | | Standard | Chi- | |
|---|---|---|---|---|---|
| Parameter | DF | Estimate | Error | Square | Pr > ChiSq |
| Intercept | 1 | −6.2303 | 0.7692 | 65.6038 | <.0001 |
| APACHE | 1 | 0.0920 | 0.0217 | 18.0320 | <.0001 |
| AGE | 1 | 0.0457 | 0.00958 | 22.7774 | <.0001 |
| Neurologic | 1 | 0.9696 | 0.3450 | 7.8958 | 0.0050 |
| CNSD | 1 | −1.3140 | 0.3337 | 15.5021 | <.0001 |
| ARDS | 1 | 2.1080 | 0.4077 | 26.7285 | <.0001 |
| DIC | 1 | 1.2307 | 0.4772 | 6.6513 | 0.0099 |
| HBD | 1 | 1.7484 | 0.6112 | 8.1821 | 0.0042 |

TABLE 5
Odds Ratio Estimate

| | Point | 95% Wald | |
|---|---|---|---|
| Effect | Estimate | Confidence Limits | |
| APACHE | 1.096 | 1.051 | 1.144 |
| AGE | 1.047 | 1.027 | 1.067 |
| Neurologic | 2.637 | 1.341 | 5.185 |
| CNSD | 0.269 | 0.140 | 0.517 |
| ARDS | 8.232 | 3.702 | 18.304 |
| DIC | 3.424 | 1.344 | 8.724 |
| HBD | 5.745 | 1.734 | 10.037 |

Baseline data of selected patient parameters from the NORASEPT II patients was then entered into the SMART profiles generated from the NORASEPT I study and the predictiveness of the SMART profiles in determining efficacy of the TNFmAb in patients with sepsis or septic shock was assessed. Survival analyses in all patients versus SMART patients are shown below:

TABLE 6
ALL SUBJECTS:
Summary of the Number of Censored and Uncensored Values

| DRUG | Total | Failed | Censored | % Censored |
|---|---|---|---|---|
| Placebo | 863 | 379 | 484 | 56.0834 |
| TNFMab | 878 | 360 | 518 | 58.9977 |
| Total | 1741 | 739 | 1002 | 57.5531 |

| TEST | Chi-Square | DF | p-value |
|---|---|---|---|
| −2logLR | 2.0472 | 1 | 0.1525 |

TABLE 7
SMART SUBJECTS: Treatment death probability 1e .6 and
Placebo Death Probability ge .3

| DRUG | Total | Failed | Censored | % Censored |
|---|---|---|---|---|
| Placebo | 371 | 184 | 187 | 50.4043 |
| TNFMab | 373 | 158 | 215 | 57.6408 |
| Total | 744 | 342 | 402 | 54.0323 |

| TEST | Chi-Square | DF | p-value |
|---|---|---|---|
| −2LogLR | 5.3601 | 1 | 0.0206 |

Thus, SMART was capable of objectively identifying, at pre-randomization baseline, individual patients who were biologically appropriate for a study drug.

Following the successful application of SMART with these treatment regimens, the method was applied to clinical trial data testing the efficacy of IL-1ra in severe sepsis or septic shock. The goal of this analysis was to demonstrate the use of SMART to identify patients most likely to respond to treatment with IL-1ra. This was done by predicting the host inflammatory response to infection at pre-randomization sepsis baseline of individual patients biologically responsive to IL-1ra and simultaneously excluding those patients enrolled in the clinical study by consensus definitions of sepsis but who would not benefit from IL-1ra. Using the previously established SMART methodology as described supra for the E5 clinical study analysis, the placebo (n=302) and the active IL-1ra groups (2.0 mg/kg/hr, n=293 and 1.0 mg/kg/hr n=298) from a Phase III randomized, double blind clinical trial of IL-1ra in sepsis were analyzed. The results of the clinical trial have been reported (Fisher et al. 1994. JAMA 271:1836-1843). Established, universally accepted clinical definitions of sepsis and septic shock were used as study entry criteria. Using clinical data collected at the baseline of sepsis onset, stepwise multivariate logistic regression with all ways elimination of independent variables was used to develop predictive models for mortality risk among placebo patients and separately for patients who received low dose IL-1ra and high dose IL-1ra. These models were then applied to the original study population, at pre-randomization baseline, by entering data from each patient into the SMART predictive models.

All available data (pre-randomization) from the IL-1ra study case report forms were tabulated. Baseline information included demographics, physiologic observations, and hospital laboratory tests. These parameters are listed in Table 8 below.

TABLE 8
Independent Variables in Patients with Severe Sepsis

| | | |
|---|---|---|
| Age | WBC | PaO₂/FiO₂ |
| Sex | IL-6 | Chloride |
| Race | IL-8 | Eosinophils |
| Albumin | GCSF | Lymphocytes |
| Alkaline | EKG: P-r and q-T | Segmental |
| phosphatase | intervals | neutrophils |
| ALT | DIC | Metamyelocyte |
| AST | GCS | Mononuclear cells |
| BUN | Hepatobiliary failure | Band neutrophil |
| Calcium | Shock | Basophils |
| Cholesterol | ARDS | Granulocytes |
| Creatinine | Renal failure | % Granulocytes |
| GGT | Coma | % Lymphocytes |
| Glucose | Alcohol abuse/cirrhosis | Eosinophils |
| Hematocrit | HIV | Lactic acid |
| MCH | Dialysis | PAOP |
| MCHC | Neutropenia | Cardiac index |
| MCV | COPD | SVR |
| Phosphorus | Solid tumor | PEEP |
| Platelet count | Hematologic malignancy | Pressure support |
| Potassium | Chronic renal failure | Respiratory rate |
| Total protein | Mechanical ventilation | Admitting service |
| PT | AaDO₂ | Trauma |
| PTT | Base deficit | Systolic BP |
| RBC | pH | Diastolic BP |
| Sodium | PaO₂ | Heart rate |
| Total bilirubin | SaO₂ | MAP |
| Triglycerides | FiO₂ | Temperature |
| Uric acid | Fluids in/out | Height/Weight |

Then, using multivariate, step-wise logistical regression with all ways elimination, SMART models were developed separately using baseline IL-1ra data from only the placebo population, and from the baseline database of the IL-1ra active drug groups (low dose and high dose). Logistic regressions were developed to determine which independent variables contributed to the dichotomous dependent variable of death. Patients were separated by treatment group such that logistical regression models were developed using patients who received the treatment IL-1ra at 11.0 mg/kg/hour, and a separate model for those who received IL-1ra at 2.0 mg/kg/hour, and another model was developed for only the placebo patients. Independent variables that were weighted contributors for the models were selected by step-wise logistic regression with all ways elimination. Statistical significance at the p<0.10 level was required for an independent variable to be included in the modeling process. The logistic regression models created three possible probabilities for each individual patient: the probability of survival for the patient receiving IL-1ra and either 11.0 mg/kg/hour or 2.0 mg/kg/hour, and the probability of survival for the patient receiving placebo. Possible cutoffs for these probabilities were examined to determine which patients would have the best survival if they received either of the dosages of IL-1ra, and which would recover independent of treatment arm if they received placebo. These cutoffs were determined through examining different cohorts of patients using Kaplan-Meier survival models. A lengthy exploration into the relationship between the placebo and IL-1ra models and their interactions with treatment effects was undertaken. Kaplan-Meier survival analyses of both IL-1ra versus placebo arms then were carried out according to the original randomization assignments from the study. IL-1ra versus placebo treatment effects on survival were evaluated separately among the patients who had complete data sets, and among the cohort comprised of patients predicted by SMART models to be biologically appropriate to respond to IL-1ra. Treatment effects of IL-1ra versus placebo in the SMART cohort on 28-day mortality then were analyzed by Kaplan-Meier statistics. Differences in distribution of baseline patient clinical characteristics were analyzed using the Chi-square equation.

The following tables present the results of the model for the placebo, high dose and low dose models, as well as the cut point analysis results for placebo/high dose and placebo/low dose.

TABLE 9
Placebo Model Parameter Estimates

| | | | Standard | Wald | |
|---|---|---|---|---|---|
| Parameter | DF | Estimate | Error | Chi-Square | Pr > ChiSq |
| Intercept | 1 | −10.7544 | 4.0541 | 7.0369 | 0.0080 |
| ards0 | 1 | −1.1262 | 0.3319 | 11.5128 | 0.0007 |
| dic0 | 1 | −1.2443 | 0.3874 | 10.3188 | 0.0013 |
| Map | 1 | 0.0262 | 0.00992 | 6.9529 | 0.0084 |
| Temp | 1 | 0.2848 | 0.1051 | 7.3464 | 0.0067 |
| Aphc | 1 | 1.1029 | 0.3002 | 13.4968 | 0.0002 |
| CUREA | 1 | −0.0220 | 0.00599 | 13.5445 | 0.0002 |
| FIO2 | 1 | −0.00582 | 0.00241 | 5.8326 | 0.0157 |

TABLE 10
Placebo Model Odds Ratio Estimates

| | | 95% Wald | |
|---|---|---|---|
| Effect | Point Estimate | Confidence | Limits |
| ards0 | 0.324 | 0.169 | 0.621 |
| dic0 | 0.288 | 0.135 | 0.616 |
| Map | 1.027 | 1.007 | 1.047 |
| Temp | 1.330 | 1.082 | 1.634 |
| Aphc | 3.013 | 1.673 | 5.427 |
| CUREA | 0.978 | 0.967 | 0.990 |
| FIO2 | 0.994 | 0.990 | 0.999 |

TABLE 11
High Dose Mode 1 Parameter Estimates

| | | | Standard | Weld | |
|---|---|---|---|---|---|
| Parameter | DF | Estimate | Error | Chi-Square | Pr > ChiSq |
| Intercept | 1 | 1.0059 | 1.0366 | 0.9416 | 0.3319 |
| Vasco | 1 | −0.7001 | 0.3223 | 4.7181 | 0.0298 |
| Age | 1 | −0.0188 | 0.00845 | 4.9413 | 0.0262 |
| Bpsys | 1 | 0.0185 | 0.00771 | 5.7755 | 0.0163 |
| respt_inf | 1 | −0.6779 | 0.2894 | 5.4860 | 0.0192 |
| ut_inf | 1 | 1.9726 | 0.6545 | 9.0828 | 0.0026 |
| CUREA | 1 | −0.0119 | 0.00511 | 5.4585 | 0.0195 |

TABLE 12
High Dose Model Odds Ratio Estimates

| | | 95% Wald | |
|---|---|---|---|
| Effect | Point Estimate | Confidence | Limits |
| Vasco | 0.497 | 0.264 | 0.934 |
| Age | 0.981 | 0.965 | 0.998 |
| bpsys | 1.019 | 1.003 | 1.034 |
| respt_inf | 0.508 | 0.288 | 0.895 |
| Ut_inf | 7.190 | 1.993 | 25.933 |
| CUREA | 0.988 | 0.978 | 0.998 |

TABLE 13
Low Dose Model Parameter Estimates

| | | | | Wald | |
|---|---|---|---|---|---|
| | | | Standard | Chi- | |
| Parameter | DF | Estimate | Error | Square | Pr > ChiSq |
| Intercept | 1 | 4.7511 | 0.7392 | 41.3114 | <.0001 |
| ards0 | 1 | −0.9736 | 0.3420 | 8.1020 | 0.0044 |
| dic0 | 1 | −1.2478 | 0.3721 | 11.2429 | 0.0008 |
| arf0 | 1 | −0.9416 | 0.3041 | 9.5848 | 0.0020 |
| Vasco | 1 | −0.7165 | 0.2959 | 5.8652 | 0.0154 |
| Age | 1 | −0.0277 | 0.00871 | 10.0961 | 0.0015 |
| PE_HEENT | 1 | −0.9364 | 0.3078 | 9.2545 | 0.0023 |
| PE_Abdomen | 1 | −0.4992 | 0.3145 | 2.5189 | 0.1125 |
| PE_Neurological | 1 | −0.4537 | 0.2889 | 2.4662 | 0.1163 |

TABLE 14
Low Dose Model Odds Ratio Estimates

| | Point | 95% Wald | |
|---|---|---|---|
| Effect | Estimate | Confidence | Limits |
| ards0 | 0.378 | 0.193 | 0.738 |
| dic0 | 0.287 | 0.138 | 0.595 |
| arf0 | 0.390 | 0.215 | 0.708 |
| Vasco | 0.488 | 0.274 | 0.872 |
| Age | 0.973 | 0.956 | 0.989 |
| PE_HEENT | 0.392 | 0.214 | 0.717 |
| PE_Abdomen | 0.607 | 0.328 | 1.124 |
| PE_Neurological | 0.635 | 0.361 | 1.119 |
| PE_Extremities_Joint | 0.568 | 0.320 | 1.009 |

TABLE 15
Cut-point results for Placebo/High dose models

| | Treat- | | | % | Chisq |
|---|---|---|---|---|---|
| | ment | Survived | Total | Survived | P-value |
| Patients with Non- | High | 203 | 289 | 70.2 | 0.2824 |
| missing | Placebo | 197 | 298 | 66.1 | |
| hd > .05 and pbo < .95 | High | 188 | 270 | 69.6 | 0.1162 |
| | Placebo | 174 | 275 | 63.3 | |
| hd > .10 and pbo < .90 | High | 161 | 239 | 67.4 | 0.0360 |
| | Placebo | 134 | 231 | 58.0 | |
| hd > .15 and pbo < .85 | High | 135 | 209 | 64.6 | 0.0757 |
| | Placebo | 112 | 200 | 56.0 | |
| hd > .20 and pbo < .80 | High | 115 | 181 | 63.5 | 0.0237 |
| | Placebo | 91 | 176 | 51.7 | |
| hd > .25 and pbo < .75 | High | 94 | 151 | 62.3 | 0.0109 |
| | Placebo | 72 | 151 | 47.7 | |
| hd > .30 and pbo < .70 | High | 80 | 123 | 65.0 | 0.0009 |
| | Placebo | 59 | 133 | 44.4 | |
| hd > .35 and pbo < .65 | High | 61 | 97 | 62.9 | 0.0007 |
| | Placebo | 42 | 107 | 39.3 | |
| hd > .40 and pbo < .60 | High | 52 | 84 | 61.9 | 0.0008 |
| | Placebo | 34 | 93 | 36.6 | |
| hd > .45 and pbo < .55 | High | 43 | 72 | 59.7 | 0.0008 |
| | Placebo | 25 | 77 | 32.5 | |
| hd > .50 and pbo < .50 | High | 36 | 56 | 64.3 | <.0001 |
| | Placebo | 16 | 63 | 25.4 | |
| hd > .55 and pbo < .45 | High | 31 | 40 | 77.5 | <.0001 |
| | Placebo | 13 | 48 | 27.1 | |
| hd > .60 and pbo < .40 | High | 20 | 27 | 74.1 | 0.0003 |
| | Placebo | 9 | 33 | 27.3 | |

TABLE 16
Cut-point results for Placebo/Low dose models

| | Treat- | | | % | Chisq |
|---|---|---|---|---|---|
| | ment | Survived | Total | Survived | P-value |
| Patients with Non- | Low | 197 | 290 | 67.9 | 0.6178 |
| missing | Placebo | 196 | 297 | 66.0 | |
| ld > .05 and pbo < .95 | Low | 179 | 271 | 66.1 | 0.4771 |
| | Placebo | 173 | 274 | 63.1 | |
| ld > .10 and pbo < .90 | Low | 151 | 237 | 63.7 | 0.2742 |
| | Placebo | 134 | 228 | 58.8 | |
| ld > .15 and pbo < .85 | Low | 128 | 197 | 65.0 | 0.0903 |
| | Placebo | 111 | 196 | 56.6 | |
| ld > .20 and pho < .80 | Low | 109 | 165 | 66.1 | 0.0171 |
| | Placebo | 90 | 169 | 53.3 | |
| ld > .25 and pbo < .75 | Low | 87 | 132 | 65.9 | 0.0093 |
| | Placebo | 71 | 141 | 50.4 | |
| ld > .30 and pbo < .70 | Low | 70 | 107 | 65.4 | 0.0055 |
| | Placebo | 57 | 121 | 47.1 | |
| ld > .35 and pbo < .65 | Low | 62 | 90 | 68.9 | 0.0003 |
| | Placebo | 41 | 96 | 42.7 | |
| ld > .40 and pbo < .60 | Low | 48 | 66 | 72.7 | <.0001 |
| | Placebo | 33 | 82 | 40.2 | |
| ld > .45 and pbo < .55 | Low | 40 | 54 | 74.1 | <.0001 |
| | Placebo | 23 | 61 | 37.7 | |
| ld > .50 and pbo < .50 | Low | 30 | 42 | 71.4 | 0.0001 |
| | Placebo | 14 | 46 | 30.4 | |
| ld > .55 and pbo < .45 | Low | 20 | 29 | 69.0 | 0.0016 |
| | Placebo | 8 | 29 | 27.6 | |
| ld > .60 and pbo < .40 | Low | 14 | 21 | 66.7 | 0.0327 |
| | Placebo | 5 | 16 | 31.3 | |

Clinically significant IL-1ra efficacy had not been reported by the original study investigators (see Fisher et al. 1994. JAMA 271:1836-1843), who observed a non-significant maximum survival benefit of only 5%. However, using the method of the present invention, statistically and clinically significant IL-1ra efficacy in meeting the primary study endpoint of reduced septic mortality was achieved in a continuum of IL-1ra potency levels. With SMART, IL-1ra lowered mortality by 9% (16% relative to placebo) in patients who received IL-1ra at 2.0 mg/kg/hour, when those with SMART predicted placebo mortality of <10% and those with predicted IL-1ra mortality >90% were excluded. IL-1ra achieved a 50.4% absolute septic mortality reduction (69% relative to placebo) when SMART predicted placebo mortality <55% and IL-1ra mortality >45% were excluded. Within this range, for both the 1.0 mg/kg/hour and 2.0 mg/kg/hour dosage regimens, IL-1ra reduced septic morality consistently, at increasing levels of efficacy as the SMART population became more IL-1ra specific.

Therefore, by using SMART methods to analyze the database of patients from the IL-1ra clinical trial, a subgroup of patients was identified where the efficacy of IL-1ra was proven. As a result, the present method can now be applied to future patients who may be in need of treatment for sepsis. IL-1ra treatment could be used after the patient's SMART profile is compared with the profile now established for IL-1ra in order to predict whether treatment would be capable of showing benefit to the patients, where benefit would be seen in terms of reduced mortality likelihood and/or increased survival time.

As a result, these applications of the SMART methodology show that the predictions can supplement clinical entry criteria for studies of antibiotics, cancer treatments, and transplant regimens, among others, as well as new drugs for sepsis, acute organ failure, and other systemic inflammatory conditions. SMART profiles ensure that the study drug receives a reasonable chance to demonstrate its efficacy in the conditions under treatment. After SMART profiling is used to demonstrate a drug's efficacy, SMART profiles can then be applied at the bedside to identify individual patients for whom the drug in question is beneficial. Using SMART, the host inflammatory response of individuals can now be matched to the biopharmacologic properties of a drug. This method is therefore a way to enhance the likelihood that clinical efficacy will be demonstrated in clinical trials.

The invention is further illustrated by the following nonlimiting examples.

### EXAMPLES

### Example 1

### Measured Physiologic Parameters from Patients with Sepsis

Physiologic parameters in nine septic patients were monitored for 4 days. Each of these patients suffered from most, if not all, of the following: a fever greater than 100.4° F.; a heart rate greater than 90 beats/minute; a respiratory rate greater than 20 breaths/minute or mechanical ventilation required; other clinical evidence to support a diagnosis of sepsis syndrome; profound systemic hypotension characterized by a systolic blood pressure of less than 90 mm mercury or a mean arterial pressure less than 70 mm mercury; clinical dysfunction of the brain, lungs, liver, or coagulation system; a hyperdynamic cardiac index and systemic vascular resistance, and systemic metabolic/lactic acidosis. Levels of thromboxane B2, prostaglandin 6-keto F1α (PGI), leukotrienes B₄, C₄, D₄ and E₄, interleukin-1β, tumor necrosis factor α, and interleukin-6 were measured serially in plasma from these patients. Leukotriene B₄ and/or tumor necrosis factor α were detectable in only two patients. Plasma levels of thromboxane B₂, PGI, and the complements of leukotrienes C₄, D₄ and E₄ were elevated above normal and increased significantly from baseline during the first 72 hours. Plasma levels of interleukin-1β did not change from baseline, however, levels of interleukin-6 rose sequentially to 118% of the baseline values. In 10 additional patients who received a 72 hour infusion of human recombinant interleukin-1 antagonist, at 72 hours thromboxane B₂, PGI, leukotrienes C₄, D₄ and E₄, and interleukin-6 plasma levels were significantly lower. Interleukin-1β was significantly increased in these patients when compared with septic patients who received only standard care. Retrospective data analysis of the overall study suggested survival benefit in patients who received the interleukin-1 antagonist which, in the sub-group studied above, had lower prostaglandin, leukotriene, and IL-6 levels and higher plasma interleukin-1.

### Example 2

### Application of the SMART Profile to Patients Enrolled in a Clinical Trial for Severe Sepsis

The purpose of this study was to demonstrate the ability of the SMART method to identify interactions among physiologic parameters, standard hospital laboratory tests, patient demographics, and circulating cytokine levels that predict continuous and dichotomous dependent clinical variables in advance in individual patients with severe sepsis and septic shock. Patients (n=303) with severe sepsis or septic shock were entered into the placebo arm of a multi-institutional clinical trial. The patients were randomly divided into a model-building training cohort (n=200) and a prospective validation or predictive cohort (n=103). Demographics, including sex, race, age, admitting service (surgery or non-surgical), and co-morbidities were recorded at baseline for each patient (Table 17). At baseline and on days 1 through 7, 14, 21, and 28, the physiologic parameters and hospital laboratory tests were recorded. In addition, at baseline and on days 1, 2, 3, and 4 plasma concentrations of interleukin-6 (IL-6), interleukin-8 (IL-8), and granulocyte colony stimulating factor (GCSF) were measured by ELISA using commercially available kits and standard ELISA methodology.

The continuous dependent variables were screened for cross-correlations with each independent variable at days 1-7, 14, 21, and 28 after baseline. Cross correlations with correlation coefficients of 0.1 or higher were then entered into a matrix program in which multiple regression models with all ways elimination were built for each continuous dependent variable for each day. In order to maintain adequate standards for statistical power, the number of independent variables included in each model was limited to approximately one for every 20 patients in each data set evaluated. These multiple regression predictive models then were validated prospectively by entering raw data from each of the patients in the predictive cohort into them and plotting linear regression curves for the predictive value of each variable for each patient versus the measurements actually observed. The extent of agreement between the quantitative predictions and observed data then was described by the Pearson product moment or linear regression correlation coefficient.

Again using the training cohort, multivariate models that predicted the presence or absence of the clinical entities such as ARDS, renal insufficiency, DIC, according to established diagnostic criteria in the literature for these entities, as well as cerebral dysfunction (Glasgow Coma Scale less than 11), and the number of lung quadrants on chest x-ray that were affected by pulmonary edema (0-4) were developed through a step-wise logistic regression. Glasgow Coma Scale less than 11 was chosen as a threshold for cerebral dysfunction because of the automatic absence of an appropriate verbal response for endotracheally intubated patients whom otherwise have intact cerebral function. The SMART multiple regression models derived for these dichotomous dependent variables were then validated prospectively by entering raw data from individual patients in the predictive cohort into the training cohort logistic regression formulae, and then assessing predictive accuracy by calculating the area under the curve (AUC) of receiver operator characteristic statistics. Multiple regression and stepwise multivariate logistic models that predicted continuous and dichotomous dependent variables, respectively, 24 hours after baseline, used baseline data only. For predictions beyond 24 hours, SMART modeling was carried out in two ways for each variable at each measured time point: 1) from baseline data only; 2) from serial data where baseline measurements and/or subsequent determinations up to 24 hours before the time being prognosticated were incorporated into the multiple regression and/or multivariate stepwise logistic regression modeling. The differences between baseline and serial predictive models were evaluated statistically using Fisher's z transformation. For this study, statistical significance was established at the 95% confidence interval with a z-statistic greater than or equal to 1.96.

Prospectively validated SMART predictions of physiologic, respiratory, and metabolic parameters in patients with severe sepsis and septic shock, resulting from multivariate models derived from baseline data only are listed in Table 18. The highest linear regression correlation coefficients were seen for predictions of the level of pressure support ventilation, PEEP, serum albumin, cholesterol, total protein, triglycerides, and uric acid. Through 7 days, quantitative predictions of HCO₃, FiO₂, SVR, cardiac index, temperature, and heart rate also approached clinically useful levels of prospective validation. Predictions from baseline data of continuous dependent variables at 14 days and beyond were consistently significant only for HCO₃, serum albumin, cholesterol, total protein, uric acid, and calcium.

Results of prospectively validated SMART multiple regression predictions of liver and renal function indicators among patients with severe sepsis from baseline data only are shown in Table 19. Clinically useful levels of correlation between SMART predictions and the values actually observed in individual patients were achieved for alkaline phosphatase, alanine aminotransferase (ALT), aspartate aminotransferase (AST), glutamyl-glutamate aminotransferase (GGT), total bilirubin, BUN, and creatinine. Many of the multiple regression models yielded clinically useful results at 14 days and beyond.

Prospectively validated SMART predictions of hematologic and coagulation indicators in patients with severe sepsis from baseline data only are tabulated in Table 20. Quantitative prediction from baseline data for lymphocyte, monocyte, segmental neutrophil, band, and granulocyte counts, and differential percentage of granulocytes and lymphocytes, platelet count, and prothrombin time (PT) consistently resulted in linear regression correlations between predicted and observed values in individual patients in the clinically useful range above 0.9. SMART predictions of hematocrit, red blood cell count (RBC), and white blood cell count (WBC), and PTT (partial thromboplastin time) also were significant.

Prospectively validated SMART predictions of physiologic, respiratory, and metabolic parameters in patients with severe sepsis from baseline data plus serial information, including maximum levels and change from baseline are tabulated in Table 21. Plots of predicted versus observed values in individual patients for Glasgow Coma Scale, HCO₃, pressure support ventilation, PEEP, albumin, cholesterol, triglycerides, and uric acid produced r values greater than 0.8 during days 1-7. Predicted versus observed correlations above 0.4 were recorded for heart rate, temperature, cardiac index, SVR, FiO₂, glucose, total protein, and calcium.

Prospective validated SMART predictions of liver and renal function indicators from baseline plus serial data are shown in Table 22. Clinically useful levels of accuracy, evidenced in Pearson product moments exceeding 0.8 were achieved with alkaline phosphatase, ALT, GGT, total bilirubin, BUN, and creatinine for up to 28 days of observation.

Prospective validated SMART predictions of hematologic and coagulation indicators in patients with severe sepsis from models derived from baseline plus serial data analysis are shown in Table 23. Clinically useful levels of accuracy were evidenced in r values exceeding 0.9 for SMART predictions of lymphocyte, monocytes, segmental neutrophil, band, and granulocyte counts, differential percentage of granulocytes and lymphocytes, platelet count, and prothrombin time. Pearson product moments exceeding 0.5 were recorded also for hematocrit, RBC, WBC, and PTT.

Predicted versus observed linear regression coefficients for continuous dependent variables in patients with severe sepsis are tabulated in Table 24. Through day 3, over half of the predicted versus observed plots of individual patients had r values at or above 0.7. For days 4 and 5, most multiple regression models were validated at or above the 0.5 level. Among predictions beyond 14 days, approximately 20% of r values were at or above 0.6. Clinically useful levels of accuracy, reflected by Pearson product moments greater than 0.8 were noted in 50% of SMART predictions for continuous dependent variables at day 1, 47% at day 2, and 25% at day 3. Thereafter, through day 28, 14 to 22% of quantitative predictions in individual patients generated predicted versus observed plots at or above the 0.8 r value level of accuracy.

The distribution of regression coefficients for prospectively validated SMART predictions of continuous dependent variables in individual patients with severe sepsis from baseline data plus serial data are listed in Table 25. Through day 5, from baseline, over half of predicted versus observed r values were greater than 0.5, and 53% had r values exceeding 0.8 at day 3 from baseline. On days 4-28, between 17% and 31% of serial data multiple regression models generated predictive versus observed Pearson product moments of 0.8 and higher.

In order to determine the ability of the SMART predictive modeling process to predict organ failure and shock subclinically in patients with severe sepsis, baseline data from patients in the predictive cohort who did not have ARDS at baseline were entered into the SMART models for predicting ARDS from baseline data on days 1-28. Similarly, data from patients who did not have DIC at baseline were entered into models for DIC and so on, as well as for individual patients who did not have hepatobiliary failure, renal insufficiency, shock, and Glasgow Coma Scale less than 11 at baseline. SMART multiple logistic regression models predicted the presence or absence of ARDS, DIC, hepatobiliary failure, renal insufficiency, shock, and cerebral dysfunction in patients without each of these conditions at baseline up to 28 days in advance with 25 of 60 (42%) achieving ROC AUC values of 0.7 and higher. Conversely, predicted versus observed analysis for shock and each type of organ dysfunction was performed using baseline data from predictive cohort patients who did have shock or organ dysfunction at baseline. In 38 of 60 models (63%), the ROC AUC for predicted versus observed plots exceeded 0.5, thus predicting the continued presence or resolution of shock and organ failure.

TABLE 17
Independent Variables in Patients with Severe
Sepsis

| | | |
|---|---|---|
| Age | WBC | PaO₂/FiO₂ |
| Sex | IL-6 | Chloride |
| Race | IL-8 | Eosinophils |
| Albumin | GCSF | Lymphocytes |
| Alkaline | EKG: P-r and q-T | Segmental |
| phosphatase | intervals | neutrophils |
| ALT | DIC | Metamyelocyte |
| AST | GCS | Mononuclear cells |
| BUN | Hepatobiliary failure | Band neutrophil |
| Calcium | Shock | Basophils |
| Cholesterol | ARDS | Granulocytes |
| Creatinine | Renal failure | % Granulocytes |
| GGT | Coma | % Lymphocytes |
| Glucose | Alcohol abuse/cirrhosis | Eosinophils |
| Hematocrit | HIV | Lactic acid |
| MCH | Dialysis | PAOP |
| MCHC | Neutropenia | Cardiac index |
| MCV | COPD | SVR |
| Phosphorus | Solid tumor | PEEP |
| Platelet count | Hematologic malignancy | Pressure support |
| Potassium | Chronic renal failure | Respiratory rate |
| Total protein | Mechanical ventilation | Admitting service |
| PT | AaDO₂ | Trauma |
| PTT | Base deficit | Systolic BP |
| RBC | pH | Diastolic BP |
| Sodium | PaO₂ | Heart rate |
| Total bilirubin | SaO₂ | MAP |
| Triglycerides | FiO₂ | Temperature |
| Uric acid | Fluids in/out | Height/Weight |

TABLE 18
Prediction of Physiologic, Respiratory and Metabolic Parameters from
Baseline Data Only

| | Day | | | | | | | | | |
|---|---|---|---|---|---|---|---|---|---|---|
| r¹ | 1 | 2 | 3 | 4 | 5 | 6 | 7 | 14 | 21 | 28 |
| Heart Rate | 0.429 | 0.425 | 0.310 | 0.249 | 0.360 | 0.386 | 0.377 | 0.109 | 0.183 | 0.366 |
| Temperature | 0.468 | 0.411 | 0.161 | 0.243 | 0.371 | 0.295 | 0.342 | 0.033 | 0.177 | — |
| Cardiac Index | 0.570 | 0.445 | 0.645 | 0.437 | 0.525 | 0.440 | — | — | — | — |
| SVR | 0.488 | 0.304 | 0.420 | −.014 | 0.061 | 0.265 | 0.124 | — | — | — |
| Glasgow Coma Scale | 0.601 | 0.575 | 0.458 | 0.387 | 0.287 | 0.400 | 0.325 | 0.184 | 0.213 | 0.101 |
| FiO₂ | 0.443 | 0.115 | 0.078 | 0.452 | 0.517 | 0.308 | 0.409 | 0.023 | 0.218 | 0.092 |
| HCO₃ | 0.571 | 0.551 | 0.562 | 0.477 | 0.500 | 0.401 | 0.350 | 0.371 | 0.421 | 0.126 |
| Pressure Support | 0.893 | 0.738 | 0.763 | 0.402 | 0.421 | 0.481 | 0.167 | — | 0.290 | — |
| PEEP | 0.893 | 0.716 | 0.669 | 0.372 | 0.391 | 0.270 | 0.317 | 0.168 | 0.016 | 0.071 |
| Albumin | 0.881 | 0.720 | 0.770 | 0.767 | 0.767 | 0.709 | 0.647 | 0.420 | 0.373 | 0.204 |
| Cholesterol | 0.725 | 0.832 | 0.794 | 0.722 | 0.479 | 0.395 | 0.295 | 0.356 | 0.258 | 0.055 |
| Glucose | 0.217 | 0.251 | 0.247 | 0.447 | 0.472 | — | 0.079 | 0.197 | 0.239 | 0.313 |
| Total Protein | 0.785 | 0.684 | 0.701 | 0.635 | 0.587 | 0.556 | 0.483 | 0.289 | 0.229 | 0.031 |
| Triglycerides | 0.711 | 0.922 | 0.771 | 0.403 | 0.407 | 0.313 | 0.155 | 0.343 | 0.194 | 0.120 |
| Uric Acid | 0.939 | 0.910 | 0.826 | 0.740 | 0.685 | 0.593 | 0.506 | 0.283 | 0.353 | 0.512 |
| Calcium | 0.696 | 0.663 | 0.424 | 0.580 | 0.611 | 0.605 | 0.510 | 0.360 | 0.450 | 0.312 |

TABLE 19
Prediction of Liver and Renal Function Indicators in Severe Sepsis
From Baseline Data Only

| | Day | | | | | | | | | |
|---|---|---|---|---|---|---|---|---|---|---|
| r¹ | 1 | 2 | 3 | 4 | 5 | 6 | 7 | 14 | 21 | 28 |
| Alkaline | 0.869 | 0.550 | 0.691 | 0.679 | 0.798 | 0.710 | 0.619 | 0.421 | 0.369 | 0.105 |
| Phosphatase | | | | | | | | | | |
| ALT | 0.959 | 0.844 | 0.391 | 0.485 | 0.606 | 0.242 | 0.224 | 0.354 | 0.305 | 0.108 |
| AST | 0.786 | 0.659 | 0.231 | 0.287 | 0.153 | 0.061 | 0.093 | — | — | 0.461 |
| GGT | 0.943 | 0.807 | 0.717 | 0.707 | 0.671 | 0.499 | 0.578 | 0.491 | 0.456 | 0.169 |
| Total Bilirubin | 0.965 | 0.941 | 0.832 | 0.676 | 0.770 | 0.753 | 0.824 | 0.869 | 0.815 | 0.688 |
| BUN | 0.970 | 0.922 | 0.881 | 0.832 | 0.816 | 0.804 | 0.767 | 0.450 | 0.337 | 0.331 |
| Creatinine | 0.896 | 0.831 | 0.741 | 0.706 | 0.657 | 0.645 | 0.567 | 0.303 | 0.384 | 0.379 |

TABLE 20
Prediction of Hematologic and Coagulation Indicators In Severe Sepsis
From Baseline Data

| | Day | | | | | | | | | |
|---|---|---|---|---|---|---|---|---|---|---|
| r¹ | 1 | 2 | 3 | 4 | 5 | 6 | 7 | 14 | 21 | 28 |
| Hematocrit | 0.512 | 0.226 | 0.297 | 0.332 | 0.514 | 0.391 | 0.378 | 0.220 | 0.417 | 0.044 |
| RBC | 0.592 | 0.371 | 0.288 | 0.310 | 0.447 | 0.354 | 0.384 | 0.075 | 0.323 | 0.119 |
| WBC | 0.726 | 0.481 | 0.259 | 0.304 | 0.041 | 0.236 | 0.317 | 0.476 | 0.242 | 0.231 |
| Lymphocytes | 0.937 | 0.982 | 0.976 | 0.994 | 0.105 | 0.158 | 0.114 | 0.995 | 0.978 | 0.980 |
| Monocytes | 0.971 | 0.998 | 0.997 | 0.994 | 0.999 | 0.161 | 0.168 | 0.988 | 0.999 | 0.997 |
| Segmental | 0.999 | 0.999 | 0.999 | 0.999 | 0.999 | 0.999 | 0.999 | 0.989 | 0.995 | 0.999 |
| Neutrophils | | | | | | | | | | |
| Bands | 0.999 | 0.991 | 0.704 | 0.511 | 0.935 | 0.994 | 0.984 | 0.020 | 0.102 | 0.331 |
| Granulocytes | 0.999 | 0.999 | 0.999 | 0.859 | 0.999 | 0.878 | 0.734 | 0.637 | 0.999 | — |
| % Granulocytes | 0.999 | 0.999 | 0.999 | 0.685 | 0.999 | 0.999 | 0.999 | 0.245 | 0.999 | — |
| % Lymphocytes | 0.116 | 0.985 | 0.977 | 0.158 | 0.100 | 0.184 | — | 0.959 | 0.979 | 0.973 |
| Platelet Count | 0.921 | 0.850 | 0.777 | 0.732 | 0.670 | 0.604 | 0.438 | 0.301 | 0.450 | 0.147 |
| PT | 0.932 | 0.923 | 0.926 | 0.922 | 0.917 | 0.402 | 0.928 | 0.879 | 0.809 | 0.887 |
| PTT | 0.482 | 0.474 | 0.483 | 0.462 | 0.232 | 0.377 | 0.215 | 0.255 | 0.169 | 0.042 |

TABLE 21
Prediction of Physiologic, Respiratory and Metabolic Parameters In Severe Sepsis
From Serial Data

| | Day | | | | | | | | | |
|---|---|---|---|---|---|---|---|---|---|---|
| r | 1 | 2 | 3 | 4 | 5 | 6 | 7 | 14 | 21 | 28 |
| Heart Rate | 0.429 | 0.424 | 0.440 | 0.123 | 0.390 | 0.364 | 0.275 | 0.111 | 0.231 | 0.353 |
| Temperature | 0.468 | 0.422 | 0.205 | 0.233 | 0.354 | 0.230 | 0.300 | 0.231 | 0.201 | 0.031 |
| Cardiac Index | 0.570 | 0.157 | 0.404 | 0.352 | 0.298 | 0.167 | 0.007 | — | — | — |
| SVR | 0.488 | 0.065 | 0.224 | 0.700 | 0.804 | 0.328 | 0.223 | — | — | — |
| Glasgow Coma Scale | 0.601 | 0.897 | 0.804 | 0.665 | 0.377 | 0.024 | 0.164 | — | 0.066 | 0.079 |
| FiO₂ | 0.443 | 0.120 | 0.078 | 0.419 | 0.336 | 0.310 | 0.382 | 0.455 | 0.137 | 0.057 |
| HCO₃ | 0.571 | 0.277 | 0.853 | 0.375 | 0.362 | 0.211 | 0.350 | 0.112 | 0.233 | 0.218 |
| Pressure Support | 0.893 | 0.877 | 0.904 | 0.674 | 0.620 | 0.481 | 0.297 | 0.258 | — | 0.325 |
| PEEP | 0.892 | 0.877 | 0.899 | 0.674 | 0.263 | 0.291 | 0.450 | 0.167 | 0.368 | 0.188 |
| Albumin | 0.881 | 0.815 | 0.937 | 0.794 | 0.819 | 0.680 | 0.622 | 0.386 | 0.227 | 0.055 |
| Cholesterol | 0.725 | 0.832 | 0.957 | 0.633 | 0.403 | 0.303 | 0.180 | 0.287 | 0.011 | 0.058 |
| Glucose | 0.217 | 0.225 | 0.407 | 0.478 | 0.437 | 0.133 | 0.024 | 0.192 | 0.223 | 0.120 |
| Total Protein | 0.785 | 0.656 | 0.638 | 0.598 | 0.588 | 0.563 | 0.520 | 0.324 | 0.047 | 0.204 |
| Triglycerides | 0.711 | 0.846 | 0.802 | 0.415 | 0.602 | 0.454 | 0.158 | 0.457 | 0.384 | 0.117 |
| Uric Acid | 0.939 | 0.910 | 0.957 | 0.720 | 0.623 | 0.545 | 0.446 | 0.304 | 0.353 | 0.517 |
| Calcium | 0.696 | 0.522 | 0.346 | 0.589 | 0.551 | 0.635 | 0.142 | 0.357 | 0.553 | 0.153 |

TABLE 22
Prediction of Liver and Renal Function Indicators in Severe Sepsis
From Serial Data

| | Day | | | | | | | | | |
|---|---|---|---|---|---|---|---|---|---|---|
| r¹ | 1 | 2 | 3 | 4 | 5 | 6 | 7 | 14 | 21 | 28 |
| Alkaline | 0.869 | 0.594 | 0.689 | 0.055 | 0.878 | 0.720 | 0.809 | 0.699 | 0.670 | 0.818 |
| Phosphatase | | | | | | | | | | |
| ALT | 0.959 | 0.865 | 0.772 | 0.506 | 0.497 | 0.175 | 0.016 | 0.041 | 0.161 | 0.572 |
| AST | 0.786 | 0.659 | 0.605 | 0.180 | 0.134 | 0.302 | — | — | 0.138 | 0.426 |
| GGT | 0.943 | 0.810 | 0.837 | 0.689 | 0.701 | 0.683 | 0.736 | 0.652 | 0.443 | 0.415 |
| Total Bilirubin | 0.965 | 0.982 | 0.983 | 0.889 | 0.895 | 0.912 | 0.822 | 0.927 | 0.949 | 0.933 |
| BUN | 0.970 | 0.970 | 0.946 | 0.906 | 0.811 | 0.844 | 0.792 | 0.419 | 0.553 | 0.429 |
| Creatinine | 0.896 | 0.879 | 0.815 | 0.716 | 0.603 | 0.593 | 0.568 | 0.312 | 0.384 | 0.359 |

TABLE 23
Prediction of Hematologic and Coagulation Indicators In Severe Sepsis
From Serial Data

| | Day | | | | | | | | | |
|---|---|---|---|---|---|---|---|---|---|---|
| r¹ | 1 | 2 | 3 | 4 | 5 | 6 | 7 | 14 | 21 | 28 |
| Hematocrit | 0.512 | 0.400 | 0.045 | 0.560 | 0.410 | 0.450 | 0.403 | 0.181 | 0.027 | 0.025 |
| RBC | 0.592 | 0.658 | 0.691 | 0.134 | 0.330 | 0.369 | 0.382 | — | 0.179 | 0.327 |
| WBC | 0.726 | 0.481 | 0.751 | 0.426 | 0.095 | 0.357 | 0.353 | 0.516 | 0.377 | 0.116 |
| Lymphocytes | 0.937 | 0.982 | 0.975 | 0.989 | 0.132 | 0.996 | 0.970 | 0.994 | 0.986 | 0.981 |
| Monocytes | 0.971 | 0.989 | 0.989 | 0.387 | 0.999 | 0.161 | 0.139 | 0.988 | 0.999 | 0.998 |
| Segmental | 0.999 | 0.999 | 0.999 | 0.999 | 0.999 | 0.999 | 0.999 | 0.999 | 0.999 | 0.999 |
| Neutrophils | | | | | | | | | | |
| Bands | 0.999 | 0.989 | 0.038 | 0.519 | 0.956 | 0.995 | 0.980 | 0.095 | 0.102 | 0.386 |
| Granulocytes | 0.999 | 0.999 | 0.999 | 0.857 | 0.999 | 0.999 | 0.748 | 0.658 | 0.999 | — |
| % Granulocytes | 0.999 | 0.999 | 0.999 | 0.704 | 0.999 | 0.999 | 0.999 | 0.209 | 0.999 | — |
| % Lymphocytes | 0.116 | 0.974 | 0.986 | 0.969 | 0.116 | 0.996 | — | 0.963 | 0.984 | 0.977 |
| Platelet Count | 0.921 | 0.894 | 0.759 | 0.754 | 0.754 | 0.789 | 0.726 | 0.382 | 0.743 | 0.581 |
| PT | 0.932 | 0.932 | 0.991 | 0.885 | 0.912 | 0.911 | 0.900 | 0.866 | 0.849 | 0.865 |
| PTT | 0.482 | 0.507 | 0.472 | 0.434 | 0.246 | 0.279 | 0.348 | 0.181 | 0.726 | — |

TABLE 24
Predicted vs. Observed Linear regression Coefficients for Continuous Dependent
Variables In Patients with Severe Sepsis From Baseline Data

| Days After | Regression Coefficient | | | | |
|---|---|---|---|---|---|
| Baseline | >0.5 | >0.6 | >0.7 | >0.8 | >0.9 |
| 1 | 29/36 (81%) | 25/36 (69%) | 22/36 (61%) | 18/36 (50%) | 13/36 (36%) |
| 2 | 26/36 (72%) | 23/36 (64%) | 20/36 (56%) | 9/36 (25%) | 7/36 (19%) |
| 3 | 22/36 (61%) | 22/36 (61%) | 19/36 (53%) | 9/36 (25%) | 7/36 (19%) |
| 4 | 18/36 (50%) | 16/36 (44%) | 12/36 (33%) | 6/36 (19%) | 4/36 (11%) |
| 5 | 21/36 (58%) | 16/36 (44%) | 10/36 (28%) | 7/36 (19%) | 6/36 (17%) |
| 6 | 13/36 (36%) | 11/36 (31%) | 8/36 (22%) | 5/36 (14%) | 3/36 (8%) |
| 7 | 13/36 (36%) | 9/36 (25%) | 7/36 (19%) | 5/36 (14%) | 4/36 (11%) |
| 14 | 7/36 (19%) | 7/36 (19%) | 6/36 (17%) | 6/36 (17%) | 4/36 (11%) |
| 21 | 8/36 (22%) | 8/36 (22%) | 8/36 (22%) | 8/36 (22%) | 6/36 (17%) |
| 28 | 6/36 (17%) | 6/36 (17%) | 5/36 (14%) | 5/36 (14%) | 4/36 (11%) |

TABLE 25
Predicted vs. Observed Linear regression Coefficients for Continuous Dependent
Variables In Patients with Severe Sepsis From Serial Data

| Days After | Regression Coefficient | | | | |
|---|---|---|---|---|---|
| Baseline | >0.5 | >0.6 | >0.7 | >0.8 | >0.9 |
| 1 | 29/36 (81%) | 25/36 (69%) | 22/36 (61%) | 18/36 (50%) | 13/36 (36%) |
| 2 | 26/36 (72%) | 23/36 (64%) | 21/36 (58%) | 21/36 (58%) | 13/36 (36%) |
| 3 | 26/36 (72%) | 26/36 (72%) | 21/36 (58%) | 19/36 (53%) | 13/36 (36%) |
| 4 | 22/36 (61%) | 13/36 (36%) | 13/36 (36%) | 7/36 (19%) | 4/36 (11%) |
| 5 | 19/36 (53%) | 17/36 (47%) | 12/36 (33%) | 11/36 (31%) | 6/36 (17%) |
| 6 | 17/36 (47%) | 14/36 (39%) | 10/36 (28%) | 9/36 (25%) | 7/36 (19%) |
| 7 | 14/36 (39%) | 12/36 (33%) | 11/36 (31%) | 7/36 (19%) | 5/36 (14%) |
| 14 | 10/36 (28%) | 9/36 (25%) | 6/36 (17%) | 6/36 (17%) | 5/36 (14%) |
| 21 | 13/36 (36%) | 11/36 (31%) | 10/36 (28%) | 8/36 (22%) | 7/36 (19%) |
| 28 | 11/36 (31%) | 10/36 (28%) | 7/36 (19%) | 7/36 (19%) | 5/36 (14%) |

### Example 3

### Multiple Imputation Analysis Modeling Via SMART

Additional SMART profiles were generated from a database of patients with severe sepsis based only upon selected physiologic variables, selected standard hospital laboratory tests and selected patient demographics. Patients were randomly separated into two sets, one to be modeled (n=200) and one to validate the created models (n=102). Logistic regression was performed to predict the outcomes of organ failure, shock, ventilation and GCS. The independent variables were chosen by stepwise selection in each of five data sets to develop, at most, five different models to choose from. To determine which of the five possible models contained the best independent variables, each set of variables was modeled with the five data sets providing five different results. The deviance (−2 log likelihood) was averaged from the five different results to compare the models. The likelihood ratio test determined the best set of variables to create the best model.

The five results for the best mode were then averaged to summarize the hosmer-lemeshow test, and the area under the ARC curve. Also, the parameter estimates were averaged in accordance with the standard analysis of multiple imputation. The final models were validated by using the same patients set aside from each of the five complete data sets. The results of the area under the ARC curve were averaged to summarize the results. Results for an ARDS model, an HBD model, a shock model, an ARF model, a GSC model, a DIC model, and a VENT model are shown in the following Tables.

Table 26 provides a summary of the best models for each day a patient could have ARDS. As shown in the table, there are five results for each day from each of the five imputed sets.

TABLE 26
ARDS Model Summary
Imputed Sets

Hosmer and Lemeshow

| | | | |
|---|---|---|---|
| chi-sq | p-values | roc | Roc |

DAY 1 (Variables used to generate profile include aado2
ards_xy peep rptvol ards0 intra_abdominal_pelvis)

| | | | | |
|---|---|---|---|---|
| 1 | 2.26 | 0.97 | 0.935 | 0.824 |
| 2 | 11.08 | 0.2 | 0.935 | 0.823 |
| 3 | 7.5 | 0.48 | 0.947 | 0.811 |
| 4 | 5.4 | 0.71 | 0.941 | 0.803 |
| 5 | 10.99 | 0.2 | 0.945 | 0.846 |
| Average | | 0.512 | 0.941 | 0.8214 |

DAY 2 (Variables used to generate profile include bmi ards0
gasti_inf urinary_tract)

| | | | | |
|---|---|---|---|---|
| 1 | 1.79 | 0.99 | 0.949 | 0.816 |
| 2 | 3.56 | 0.89 | 0.951 | 0.823 |
| 3 | 5.22 | 0.73 | 0.944 | 0.819 |
| 4 | 3.99 | 0.86 | 0.949 | 0.821 |
| 5 | 3.43 | 0.9 | 0.947 | 0.83 |
| Average | | 0.874 | 0.948 | 0.8218 |

DAY 3 (Variables used to generate profile include peep pe_heent
ards0 gasti_inf)

| | | | | |
|---|---|---|---|---|
| 1 | 8.23 | 0.41 | 0.903 | 0.807 |
| 2 | 2.97 | 0.89 | 0.869 | 0.814 |
| 3 | 5.37 | 0.61 | 0.91 | 0.816 |
| 4 | 5.36 | 0.72 | 0.899 | 0.798 |
| 5 | 7.32 | 0.4 | 0.896 | 0.816 |
| Average | | 0.606 | 0.895 | 0.8096 |

DAY 4 (Variables used to generate profile include albun bmi
pe_heent ards0 arf0 gasti_inf lad pulse uflpvc)

| | | | | |
|---|---|---|---|---|
| 1 | 4.24 | 0.83 | 0.964 | 0.722 |
| 2 | 6.28 | 0.62 | 0.961 | 0.714 |
| 3 | 4.6 | 0.8 | 0.961 | 0.734 |
| 4 | 5.1 | 0.74 | 0.96 | 0.717 |
| 5 | 6.2 | 0.63 | 0.96 | 0.734 |
| Average | | 0.724 | 0.961 | 0.7242 |

DAY 5 (Variables used to generate profile include albun
endocrine_metabolic pe_heent ufin24 ards0 gasti_inf
lad)

| | | | | |
|---|---|---|---|---|
| 1 | 6.1 | 0.64 | 0.946 | 0.717 |
| 2 | 7.8 | 0.45 | 0.945 | 0.706 |
| 3 | 3.5 | 0.9 | 0.947 | 0.712 |
| 4 | 2.4 | 0.97 | 0.941 | 0.7 |
| 5 | 13.1 | 0.11 | 0.939 | 0.718 |
| Average | | 0.614 | 0.944 | 0.7106 |

DAY 6 (Variables used to generate profile include albun
endocrine_metabolic pe_heent ards0 gasti_inf uflpvc)

| | | | | |
|---|---|---|---|---|
| 1 | 10.3 | 0.25 | 0.924 | 0.757 |
| 2 | 7.4 | 0.49 | 0.916 | 0.751 |
| 3 | 8.1 | 0.43 | 0.922 | 0.765 |
| 4 | 7.6 | 0.37 | 0.916 | 0.741 |
| 5 | 8.3 | 0.4 | 0.919 | 0.754 |
| Average | | 0.388 | 0.92 | 0.7536 |

DAY 7 (Variables used to generate profile include curea
endocrine_metabolic ufin24 ards0 gasti_inf
lungcanc_xy)

| | | | | |
|---|---|---|---|---|
| 1 | 3.6 | 0.9 | 0.914 | 0.686 |
| 2 | 5.2 | 0.73 | 0.922 | 0.685 |
| 3 | 1.8 | 0.99 | 0.935 | 0.691 |
| 4 | 4.8 | 0.78 | 0.916 | 0.674 |
| 5 | 3.1 | 0.93 | 0.949 | 0.69 |
| Average | | 0.866 | 0.927 | 0.6852 |

TABLE 27
HBD Model Summary
Imputed Sets

Hosmer and Lemeshow

| | | | |
|---|---|---|---|
| chi-sq | p-values | roc | Roc |

DAY 1 (Variables used to generate profile ctbil unknown
hbd0)

| | | | | |
|---|---|---|---|---|
| 1 | 5.8 | 0.66 | 0.881 | 0.791 |
| 2 | 9.7 | 0.29 | 0.883 | 0.781 |
| 3 | 3.7 | 0.88 | 0.882 | 0.812 |
| 4 | 2 | 0.98 | 0.885 | 0.817 |
| 5 | 4.8 | 0.77 | 0.879 | 0.817 |
| Average | | 0.716 | 0.882 | 0.800 |

DAY 2 (Variables used to generate profile include blood
curea qt rptvol renal wbc hbd0)

| | | | | |
|---|---|---|---|---|
| 1 | 7.4 | 0.48 | 0.884 | 0.696 |
| 2 | 7.9 | 0.45 | 0.876 | 0.705 |
| 3 | 4.6 | 0.8 | 0.870 | 0.702 |
| 4 | 3 | 0.93 | 0.882 | 0.707 |
| 5 | 8.7 | 0.37 | 0.875 | 0.707 |
| Average | | 0.606 | 0.877 | 0.703 |

DAY 3 (Variables used to generate profile include hwbc mchc
pe_extremities_joints pe_heent pe_neurological hbd0
skin_wound)

| | | | | |
|---|---|---|---|---|
| 1 | 3.7 | 0.88 | 0.916 | 0.715 |
| 2 | 3.9 | 0.86 | 0.915 | 0.708 |
| 3 | 6.7 | 0.58 | 0.908 | 0.715 |
| 4 | 2.7 | 0.95 | 0.912 | 0.715 |
| 5 | 1.4 | 0.99 | 0.923 | 0.720 |
| Average | | 0.848 | 0.915 | 0.715 |

DAY 4 (Variables used to generate profile include apco2
cardiovascular fio2 pe_skin_appearance unknown hbd0)

| | | | | |
|---|---|---|---|---|
| 1 | 15.7 | 0.05 | 0.854 | 0.715 |
| 2 | 16.3 | 0.04 | 0.855 | 0.717 |
| 3 | 16.4 | 0.04 | 0.854 | 0.714 |
| 4 | 16.5 | 0.04 | 0.855 | 0.710 |
| 5 | 18.2 | 0.02 | 0.855 | 0.715 |
| Average | | 0.038 | 0.855 | 0.714 |

DAY 5 (Variables used to generate profile include
pe_neurological hbd0)

| | | | | |
|---|---|---|---|---|
| 1 | 3.7 | 0.88 | 0.916 | 0.715 |
| 2 | 3.9 | 0.86 | 0.915 | 0.708 |
| 3 | 6.7 | 0.56 | 0.908 | 0.715 |
| 4 | 2.7 | 0.95 | 0.912 | 0.715 |
| 5 | 1.4 | 0.99 | 0.923 | 0.720 |
| Average | | 0.848 | 0.915 | 0.715 |

DAY 6 (Variables used to generate profile include apco2
ctbil hbd0)

| | | | | |
|---|---|---|---|---|
| 1 | 0.003 | 0.99 | 0.767 | 0.768 |
| 2 | 0.003 | 0.99 | 0.767 | 0.722 |
| 3 | 0.03 | 0.99 | 0.767 | 0.768 |
| 4 | 0.003 | 0.99 | 0.767 | 0.768 |
| 5 | 0.003 | 0.99 | 0.767 | 0.753 |
| Average | | 0.99 | 0.767 | 0.756 |

DAY 7 (Variables used to generate profile include apo2 asat
bmi pe_heent pe_neurological pe_skin_appearance hbd0)

| | | | | |
|---|---|---|---|---|
| 1 | 10.7 | 0.24 | 0.848 | 0.650 |
| 2 | 5.3 | 0.72 | 0.883 | 0.665 |
| 3 | 3.8 | 0.87 | 0.869 | 0.547 |
| 4 | 8.8 | 0.36 | 0.851 | 0.649 |
| 5 | 5.7 | 0.68 | 0.880 | 0.653 |
| Average | | 0.574 | 0.866 | 0.653 |

TABLE 28
SHOCK Model Summary
Imputed Sets

Hosmer and Lemeshow

| | | | |
|---|---|---|---|
| chi-sq | p-values | roc | Roc |

DAY 1 (Variables used to generate profile include
PE_Other_body_region vasco)

| | | | | |
|---|---|---|---|---|
| 1 | 0.55 | 0.45 | 0.710 | 0.665 |
| 2 | 0.57 | 0.45 | 0.710 | 0.665 |
| 3 | 0.57 | 0.45 | 0.710 | 0.665 |
| 4 | 0.57 | 0.45 | 0.710 | 0.665 |
| 5 | 0.57 | 0.45 | 0.710 | 0.665 |
| Average | | | | |

DAY 2 (Variables used to generate profile include albun
ctbil gsc1 hgb lahb map)

| | | | | |
|---|---|---|---|---|
| 1 | 7.3 | 0.51 | 0.734 | 0.609 |
| 2 | 7.1 | 0.52 | 0.751 | 0.613 |
| 3 | 7.5 | 0.49 | 0.759 | 0.628 |
| 4 | 7.4 | 0.49 | 0.752 | 0.607 |
| 5 | 10.9 | 0.21 | 0.764 | 0.632 |
| Average | | 0.444 | 0.752 | 0.617 |

DAY 3 (Variables used to generate profile include vrate
map)

| | | | | |
|---|---|---|---|---|
| 1 | 4.1 | 0.85 | 0.734 | 0.570 |
| 2 | 2.4 | 0.97 | 0.728 | 0.568 |
| 3 | 2.7 | 0.95 | 0.737 | 0.587 |
| 4 | 3.5 | 0.9 | 0.737 | 0.563 |
| 5 | 4.5 | 0.81 | 0.741 | 0.572 |
| Average | | 0.896 | 0.735 | 0.572 |

DAY 4 (Variables used to generate profile include curea
pe_abdomen uomlkh map xyabnormal)

| | | | | |
|---|---|---|---|---|
| 1 | 7.9 | 0.45 | 0.803 | 0.606 |
| 2 | 7.4 | 0.31 | 0.796 | 0.606 |
| 3 | 8.9 | 0.35 | 0.794 | 0.576 |
| 4 | 4.3 | 0.83 | 0.806 | 0.614 |
| 5 | 5.2 | 0.74 | 0.793 | 0.594 |
| Average | | 0.536 | 0.798 | 0.599 |

DAY 5 (Variables used to generate profile include alveolar
mcv oldmi albun)

| | | | | |
|---|---|---|---|---|
| 1 | 11.7 | 0.18 | 0.740 | 0.669 |
| 2 | 10.4 | 0.24 | 0.764 | 0.708 |
| 3 | 5.4 | 0.91 | 0.777 | 0.697 |
| 4 | 15.2 | 0.06 | 0.790 | 0.671 |
| 5 | 3.5 | 0.9 | 0.748 | 0.691 |
| Average | | 0.454 | 0.764 | 0.687 |

DAY 6 (Variables used to generate profile include wbc
respt_inf)

| | | | | |
|---|---|---|---|---|
| 1 | 6.1 | 0.64 | 0.653 | 0.544 |
| 2 | 7.7 | 0.36 | 0.660 | 0.552 |
| 3 | 5.3 | 0.63 | 0.649 | 0.565 |
| 4 | 7.2 | 0.41 | 0.643 | 0.552 |
| 5 | 6.9 | 0.55 | 0.663 | 0.549 |
| Average | | 0.518 | 0.654 | 0.552 |

DAY 7 (Variables used to generate profile include albun
alveolar cirrhosis_mf_inf curea gsc1 hwbc rtrr
foreign_body_cat)

| | | | | |
|---|---|---|---|---|
| 1 | 0.63 | 0.9 | 0.626 | 0.617 |
| 2 | 0.38 | 0.94 | 0.615 | 0.615 |
| 3 | 0.73 | 0.67 | 0.625 | 0.613 |
| 4 | 0.42 | 0.93 | 0.620 | 0.611 |
| 5 | 0.66 | 0.88 | 0.620 | 0.617 |
| Average | | 0.904 | 0.621 | 0.615 |

TABLE 29
ARF Model Summary
Imputed Sets

Hosmer and
Lemeshow

| | | | |
|---|---|---|---|
| chi-sq | p-values | roc | Roc |

DAY 1 (Variables used to generate profile include apao2
icu_inf arf0 mvent)

| | | | | |
|---|---|---|---|---|
| 1 | 0.957 | 0.8 | 0.957 | 0.776 |
| 2 | 0.959 | 0.82 | 0.959 | 0.776 |
| 3 | 0.957 | 0.81 | 0.957 | 0.774 |
| 4 | 0.959 | 0.81 | 0.959 | 0.773 |
| 5 | 0.958 | 0.78 | 0.958 | 0.776 |
| Average | | 0.804 | 0.958 | 0.775 |

DAY 2 (Variables used to generate profile include ccreat
hepatic_biliary pr arf0 diffuse_xy
intra_abdominal_pelvis)

| | | | | |
|---|---|---|---|---|
| 1 | 9.8 | 0.28 | 0.943 | 0.842 |
| 2 | 9.5 | 0.3 | 0.943 | 0.828 |
| 3 | 10.1 | 0.26 | 0.944 | 0.831 |
| 4 | 11.3 | 0.19 | 0.944 | 0.836 |
| 5 | 12.1 | 0.15 | 0.944 | 0.828 |
| Average | | 0.236 | 0.944 | 0.833 |

DAY 3 (Variables used to generate profile include fio2
hepatic_biliary pr rstrol art0 dic0 resp respt_inf
tracing)

| | | | | |
|---|---|---|---|---|
| 1 | 0.69 | 0.99 | 0.975 | 0.887 |
| 2 | 4 | 0.78 | 0.966 | 0.890 |
| 3 | 2.5 | 0.96 | 0.965 | 0.882 |
| 4 | 3.6 | 0.89 | 0.965 | 0.884 |
| 5 | 2.7 | 0.95 | 0.962 | 0.888 |
| Average | | 0.914 | 0.967 | 0.882 |

DAY 4 (Variables used to generate profile include
hepatic_biliary arf0 dic0 tracing)

| | | | | |
|---|---|---|---|---|
| 1 | 1.7 | 0.8 | 0.880 | 0.843 |
| 2 | 1.7 | 0.8 | 0.880 | 0.843 |
| 3 | 1.7 | 0.8 | 0.880 | 0.843 |
| 4 | 1.7 | 0.8 | 0.880 | 0.843 |
| 5 | 1.7 | 0.8 | 0.880 | 0.843 |
| Average | | 0.8 | 0.880 | 0.843 |

DAY 5 (Variables used to generate profile include
hepatic_biliary pneum_xy arf0 foreign_body_catheter)

| | | | | |
|---|---|---|---|---|
| 1 | 2.9 | 0.71 | 0.888 | 0.818 |
| 2 | 2.9 | 0.72 | 0.888 | 0.826 |
| 3 | 2.9 | 0.71 | 0.888 | 0.826 |
| 4 | 2.9 | 0.71 | 0.888 | 0.824 |
| 5 | 2.9 | 0.74 | 0.888 | 0.820 |
| Average | | 0.718 | 0.888 | 0.823 |

DAY 6 (Variables used to generate profile include arf0
diffuse_xy)

| | | | | |
|---|---|---|---|---|
| 1 | 0.89 | 0.96 | 0.862 | 0.738 |
| 2 | 0.09 | 0.96 | 0.862 | 0.738 |
| 3 | 0.11 | 0.95 | 0.863 | 0.738 |
| 4 | 0.09 | 0.96 | 0.862 | 0.738 |
| 5 | 0.11 | 0.95 | 0.863 | 0.738 |
| Average | | 0.956 | 0.862 | 0.738 |

DAY 7

| | | | | |
|---|---|---|---|---|
| 1 | 4.4 | 0.49 | 0.886 | 0.739 |
| 2 | 3.4 | 0.49 | 0.886 | 0.739 |
| 3 | 3.4 | 0.49 | 0.886 | 0.739 |
| 4 | 3.4 | 0.49 | 0.886 | 0.739 |
| 5 | 3.4 | 0.49 | 0.886 | 0.739 |
| Average | | 0.49 | 0.886 | 0.739 |

TABLE 30
GSC Model Summary
Imputed Sets

Hosmer and
Lemeshow

| | | | |
|---|---|---|---|
| chi-sq | p-values | roc | Roc |

DAY 3 (Variables used to generate profile include csod gsc1
pedema_xy)

| | | | | |
|---|---|---|---|---|
| 1 | 0.97 | 0.280 | 0.860 | 0.710 |
| 2 | 806 | 0.360 | 0.907 | 0.692 |
| 3 | 12.8 | 0.120 | 0.917 | 0.734 |
| 4 | 33.1 | 0.000 | 0.874 | 0.727 |
| 5 | 14 | 0.080 | 0.869 | 0.700 |
| Average | | 0.168 | 0.885 | 0.713 |

DAY 4 (Variables used to generate profile include abd gsc1 pt)

| | | | | |
|---|---|---|---|---|
| 1 | 10.8 | 0.210 | 0.878 | 0.727 |
| 2 | 10 | 0.860 | 0.881 | 0.742 |
| 3 | 6.5 | 0.590 | 0.923 | 0.694 |
| 4 | 24 | 0.002 | 0.887 | 0.699 |
| 5 | 13 | 0.130 | 0.891 | 0.719 |
| Average | | 0.358 | 0.692 | 0.716 |

DAY 5 (Variables used to generate profile include fio2 gsc1
weight dic0 oldmi)

| | | | | |
|---|---|---|---|---|
| 1 | 6.5 | 0.600 | 0.883 | 0.650 |
| 2 | 11 | 0.200 | 0.892 | 0.650 |
| 3 | 6.1 | 0.630 | 0.818 | 0.714 |
| 4 | 5.7 | 0.680 | 0.883 | 0.640 |
| 5 | 5 | 0.750 | 0.893 | 0.684 |
| Average | | 0.572 | 0.874 | 0.668 |

DAY 7 (Variables used to generate profile include dic0)

| | | | | |
|---|---|---|---|---|
| 1 | NEI | NEI | 0.644 | 0.718 |
| 2 | | | 0.644 | 0.718 |
| 3 | | | 0.644 | 0.718 |
| 4 | | | 0.644 | 0.718 |
| 5 | | | 0.644 | 0.718 |
| Average | | | 0.644 | 0.718 |

NEI = Not enough information to general results

TABLE 31
DIC Model Summary
Imputed Sets

Hosmer and
Lemeshow

| | | | |
|---|---|---|---|
| chi-sq | p-values | roc | Roc |

DAY 1 (Variables used to generate profile include fio2 dic0
lbbb mfmpvc rvh temp)

| | | | | |
|---|---|---|---|---|
| 1 | 5.9 | 0.66 | 0.907 | 0.748 |
| 2 | 3.1 | 0.92 | 0.907 | 0.748 |
| 3 | 3.1 | 0.93 | 0.907 | 0.748 |
| 4 | 3.1 | 0.93 | 0.907 | 0.748 |
| 5 | 3.1 | 0.93 | 0.907 | 0.748 |
| Average | | 8.74 | 0.907 | 0.748 |

DAY 2 (Variables used to generate profile include fio2 hgb
qt utotml dic0 temp)

| | | | | |
|---|---|---|---|---|
| 1 | 4.9 | 0.77 | 0.889 | 0.896 |
| 2 | 8.1 | 0.42 | 0.866 | 0.865 |
| 3 | 6 | 0.65 | 0.888 | 0.881 |
| 4 | 7.23 | 0.51 | 0.898 | 0.761 |
| 5 | 4.2 | 0.84 | 0.892 | 0.892 |
| Average | | 0.638 | 0.887 | 0.855 |

DAY 3 (Variables used to generate profile include csod
curea fio2 dic0 rad wnd_inf)

| | | | | |
|---|---|---|---|---|
| 1 | 6.2 | 0.62 | 0.865 | 0.657 |
| 2 | 8.4 | 0.39 | 0.864 | 0.667 |
| 3 | 6.2 | 0.62 | 0.086 | 0.657 |
| 4 | 6.2 | 0.62 | 0.865 | 0.657 |
| 5 | 8.4 | 0.39 | 0.865 | 0.657 |
| Average | | 0.528 | 0.709 | 0.657 |

DAY 4 (Variables used to generate profile include fio2
renal uomlkh dic0)

| | | | | |
|---|---|---|---|---|
| 1 | 13.2 | 0.1 | 0.887 | 0.521 |
| 2 | 9.1 | 0.33 | 0.892 | 0.543 |
| 3 | 2.6 | 0.96 | 0.885 | 0.546 |
| 4 | 2.3 | 0.97 | 0.885 | 0.585 |
| 5 | 8.5 | 0.38 | 0.883 | 0.603 |
| Average | | 0.548 | 0.886 | 0.560 |

DAY 5 (Variables used to generate profile include renal
afio (only 6 had dic)

| | | | | |
|---|---|---|---|---|
| 1 | 0.27 | 0.6 | 0.839 | 0.413 |
| 2 | 0.27 | 0.6 | 0.839 | 0.413 |
| 3 | 0.27 | 0.6 | 0.839 | 0.413 |
| 4 | 0.27 | 0.6 | 0.839 | 0.413 |
| 5 | 0.27 | 0.6 | 0.839 | 0.413 |
| Average | | 0.6 | 0.839 | 0.413 |

DAY 6 (Variables used to generate profile include dic0
pulse temp height renal blood)

| | | | | |
|---|---|---|---|---|
| 1 | 9.4 | 0.31 | 0.854 | 0.642 |
| 2 | 12.7 | 0.12 | 0.853 | 0.622 |
| 3 | 19.9 | 0.01 | 0.859 | 0.627 |
| 4 | 3.7 | 0.89 | 0.890 | 0.669 |
| 5 | 21.8 | 0.005 | 0.855 | 0.619 |
| Average | | 0.267 | 0.862 | 0.636 |

DAY 7 (Variables used to generate profile include dic0
uomikh wbc)

| | | | | |
|---|---|---|---|---|
| 1 | 1.6 | 0.98 | 0.950 | 0.773 |
| 2 | 0.7 | 0.99 | 0.966 | 0.767 |
| 3 | 1.7 | 0.99 | 0.950 | 0.793 |
| 4 | 2.1 | 0.98 | 0.958 | 0.806 |
| 5 | 2.1 | 0.98 | 0.966 | 0.790 |
| Average | | 0.984 | 0.958 | 0.786 |

TABLE 32
VENT Model Summary
Imputed Sets

Hosmer and
Lemeshow

| | | | |
|---|---|---|---|
| chi-sq | p-values | roc | Roc |

DAY 1 (Variables used to generate profile include ccreat
hbd0 mvent)

| | | | | |
|---|---|---|---|---|
| 1 | 5.4 | 0.72 | 0.967 | 0.809 |
| 2 | 5.4 | 0.72 | 0.967 | 0.809 |
| 3 | 5.4 | 0.72 | 0.967 | 0.809 |
| 4 | 5.4 | 0.72 | 0.967 | 0.809 |
| 5 | 5.4 | 0.72 | 0.967 | 0.809 |
| Average | | 0.72 | 0.967 | 0.809 |

DAY 2 (Variables used to generate profile include abd arf0
emphysema hbd0 mvent pulse)

| | | | | |
|---|---|---|---|---|
| 1 | 5.4 | 0.72 | 0.871 | 0.790 |
| 2 | 7.2 | 0.51 | 0.865 | 0.766 |
| 3 | 5.4 | 0.71 | 0.858 | 0.799 |
| 4 | 3.7 | 0.88 | 0.874 | 0.764 |
| 5 | 9.2 | 0.34 | 0.872 | 0.809 |
| Average | | 0.632 | 0.868 | 0.786 |

DAY 3 (Variables used to generate profile include apco2
apo2 asat hbd0 mvent)

| | | | | |
|---|---|---|---|---|
| 1 | 4.8 | 0.77 | 0.835 | 0.800 |
| 2 | 7.4 | 0.49 | 0.852 | 0.804 |
| 3 | 10.8 | 0.21 | 0.852 | 0.782 |
| 4 | 8.7 | 0.37 | 0.851 | 0.791 |
| 5 | 5.4 | 0.71 | 0.844 | 0.790 |
| Average | | 0.51 | 0.847 | 0.793 |

DAY 4 (Variables used to generate profile include apo2
curea hbd0 mvent)

| | | | | |
|---|---|---|---|---|
| 1 | 7.5 | 0.84 | 0.807 | 0.702 |
| 2 | 7.5 | 0.84 | 0.807 | 0.705 |
| 3 | 7.5 | 0.84 | 0.807 | 0.705 |
| 4 | 7.5 | 0.84 | 0.807 | 0.705 |
| 5 | 7.5 | 0.84 | 0.807 | 0.706 |
| Average | | 0.84 | 0.807 | 0.705 |

DAY 5 (Variables used to generate profile include gsc1
respiratory ards0 gast_inf mvent resp)

| | | | | |
|---|---|---|---|---|
| 1 | 28.4 | 0.0004 | 0.833 | 0.770 |
| 2 | 21.8 | 0.005 | 0.840 | 0.771 |
| 3 | 18.7 | 0.02 | 0.832 | 0.778 |
| 4 | 25 | 0.0002 | 0.837 | 0.757 |
| 5 | 13.5 | 0.09 | 0.852 | 0.764 |
| Average | | 0.02348 | 0.839 | 0.768 |

DAY 6 (Variables used to generate profile include gsc1
hepatic_biliary peep bpdia mvent pulse)

| | | | | |
|---|---|---|---|---|
| 1 | 3 | 0.93 | 0.816 | 0.723 |
| 2 | 6.6 | 0.58 | 0.812 | 0.701 |
| 3 | 9.2 | 0.32 | 0.841 | 0.724 |
| 4 | 7.3 | 0.51 | 0.832 | 0.699 |
| 5 | 4.4 | 0.82 | 0.822 | 0.733 |
| Average | | 0.632 | 0.825 | 0.716 |

DAY 7 (Variables used to generate profile include rtrr
ards0 respt_inf)

| | | | | |
|---|---|---|---|---|
| 1 | 3.3 | 0.86 | 0.778 | 0.495 |
| 2 | 6.9 | 0.44 | 0.780 | 0.495 |
| 3 | 6 | 0.64 | 0.789 | 0.495 |
| 4 | 5.7 | 0.57 | 0.784 | 0.481 |
| 5 | 6.3 | 0.5 | 0.722 | 0.496 |
| Average | | 0.602 | 0.771 | 0.492 |

TABLE 33
DIC Model Summary
Imputed Sets

Hosmer and
Lemeshow

| | | | |
|---|---|---|---|
| chi-sq | p-values | roc | Roc |

DAY 1 (Variables used to generate profile include fio2 dic0
lbbb mfmpvc rvh temp)

| | | | | |
|---|---|---|---|---|
| 1 | 5.9 | 0.66 | 0.907 | 0.748 |
| 2 | 3.1 | 0.92 | 0.907 | 0.748 |
| 3 | 3.1 | 0.93 | 0.907 | 0.748 |
| 4 | 3.1 | 0.93 | 0.907 | 0.748 |
| 5 | 3.1 | 0.93 | 0.907 | 0.748 |
| Average | | 0.874 | 0.907 | 0.748 |

DAY 2 (Variables used to generate profile include fio2 hgb
qt utotml dic0 temp)

| | | | | |
|---|---|---|---|---|
| 1 | 4.9 | 0.77 | 0.889 | 0.896 |
| 2 | 8.1 | 0.42 | 0.866 | 0.865 |
| 3 | 6 | 0.65 | 0.888 | 0.861 |
| 4 | 7.23 | 0.51 | 0.898 | 0.751 |
| 5 | 4.2 | 0.84 | 0.892 | 0.892 |
| Average | | 0.638 | 0.887 | 0.855 |

DAY 3 (Variables used to generate profile include csod
curea fio2 dic0 rad wnd_inf)

| | | | | |
|---|---|---|---|---|
| 1 | 6.2 | 0.62 | 0.865 | 0.657 |
| 2 | 8.4 | 0.39 | 0.864 | 0.657 |
| 3 | 6.2 | 0.62 | 0.086 | 0.657 |
| 4 | 6.2 | 0.62 | 0.865 | 0.657 |
| 5 | 8.4 | 0.39 | 0.865 | 0.657 |
| Average | | 0.528 | 0.709 | 0.657 |

DAY 4 (Variables used to generate profile include fio2
renal uomlkh dic0)

| | | | | |
|---|---|---|---|---|
| 1 | 13.2 | 0.1 | 0.887 | 0.521 |
| 2 | 9.1 | 0.33 | 0.892 | 0.543 |
| 3 | 2.6 | 0.96 | 0.885 | 0.546 |
| 4 | 2.3 | 0.97 | 0.885 | 0.585 |
| 5 | 8.5 | 0.38 | 0.883 | 0.603 |
| Average | | 0.548 | 0.886 | 0.560 |

DAY 5 (Variables used to generate profile include renal
afib (only 6 had dic))

| | | | | |
|---|---|---|---|---|
| 1 | 0.27 | 0.6 | 0.839 | 0.413 |
| 2 | 0.27 | 0.6 | 0.839 | 0.413 |
| 3 | 0.27 | 0.6 | 0.839 | 0.413 |
| 4 | 0.27 | 0.6 | 0.839 | 0.413 |
| 5 | 0.27 | 0.6 | 0.839 | 0.413 |
| Average | | 0.6 | 0.839 | 0.413 |

DAY 6 (Variables used to generate profile include dic0
pulse temp height renal blood)

| | | | | |
|---|---|---|---|---|
| 1 | 9.4 | 0.31 | 0.854 | 0.642 |
| 2 | 12.7 | 0.12 | 0.853 | 0.622 |
| 3 | 19.9 | 0.01 | 0.859 | 0.627 |
| 4 | 3.7 | 0.89 | 0.890 | 0.669 |
| 5 | 21.8 | 0.005 | 0.855 | 0.619 |
| Average | | 0.267 | 0.862 | 0.636 |

DAY 7 (Variables used to generate profile include dic0
uomlkh wbc)

| | | | | |
|---|---|---|---|---|
| 1 | 1.6 | 0.98 | 0.950 | 0.773 |
| 2 | 0.7 | 0.99 | 0.966 | 0.767 |
| 3 | 1.7 | 0.99 | 0.950 | 0.793 |
| 4 | 2.1 | 0.98 | 0.958 | 0.806 |
| 5 | 2.1 | 0.98 | 0.966 | 0.790 |
| Average | | 0.984 | 0.958 | 0.786 |

### Example 4

### E5 Anti-Endotoxin Antibody Responsiveness Identified Via SMART

Independent baseline, pre-randomized variables in the final SMART profile that identified patients who were appropriate biologically for E5 are provided in Table 34. Demographic analysis and clinical observations among the 759 consensus definition patients and the 388 SMART subjects are provided in Table 35. Differences in sex and race were not significant.

TABLE 34
Independent variables

| | Odds | 95% Wald |
|---|---|---|
| | Ratio | Confidence |
| Variable | Estimates | Limits |
| Apache II | 1.039 | 1.144 |
| Urinary Tract Source of | 0.222 | 0.727 |
| Infection | | |
| Lung Source of Infection | 0.920 | 4.889 |
| Respiratory Rate | 1.008 | 1.071 |
| Diastolic Blood Pressure | 0.951 | 0.987 |
| DIC | 1.344 | 16.808 |
| Age | 1.027 | 1.067 |
| Neurologic Co-Morbidity | 1.344 | 5.185 |
| Acute Central Nervous Sys | 1.027 | 0.517 |
| Dysfunction | | |
| ARDS | 3.702 | 18.304 |
| Hepatobiliary Dysfunction | 1.734 | 19.037 |

TABLE 35
Demographic and Clinical Observations

| | | Consensus | | | |
|---|---|---|---|---|---|
| | | Criteria | | SMART Cohort | |
| | | Placebo | E5 | Placebo | E5 |
| Sex: | Men | 54% | 54% | 57% | 60% |
| | Women | 46% | 43% | 40% | 40% |
| Race: | Native American | 1.3% | 1.3% | 2.0% | 1.5% |
| | Asian | 1.0% | 0.7% | 54% | 0.05% |
| | African American | 24% | 24% | 28% | 25% |
| | Caucasian | 70% | 69% | 63% | 66% |
| | Hispanic | 5.7% | 4.9% | 5.8% | 6.5% |
| | Other | 0.8% | 0.6% | 0.5% | 0.5% |

| | | | | |
|---|---|---|---|---|
| Baseline Organ Failure | | | | |
| ARDS | 11% | 12% | 1.1% | 3.0% |
| Renal | 13.5% | 12% | 8.0% | 9.0% |
| CNS | 44% | 41% | 47% | 38% |
| DIC | 10.6% | 7.7% | 2.7% | 1.5% |
| Hepatobility | 7.6% | 4.4% | 54% | 0.5% |
| Shock | 1.6% | 1.0% | 0 | 0.5% |
| Gram Negative | 35% | 35% | 41% | 42% |
| Infection | | | | |

What is claimed is:

1. A method for analyzing clinical trial results for a new therapeutic agent comprising
a) measuring baseline parameters of a patient in a clinical trial receiving a new therapeutic agent;
b) measuring baseline parameters of a patient in the clinical trial receiving a placebo;
c) measuring outcome of the clinical trial for the patients of steps a) and b);
d) generating, with one or more of the measured parameters, a systemic mediator-associated response test profile for the patients of steps a) and b); and
e) predicting, based upon the patient profiles, what the response of each patient to the new therapeutic agent would have been prior to commencing the clinical trial thereby analyzing the clinical trial results of the new therapeutic agent.

2. The method of claim 1 wherein the new therapeutic agent is recombinant interleukin-1 receptor antagonist.
